SEQUENCE LISTING (171 sequences)

SEQ ID NO: 1 - protein, 28 aa, Homo sapiens
AKISFFFALCGFWQICHIKKHFQTHKLL

SEQ ID NO: 2 - protein, 22 aa, Homo sapiens
INYCQKKLMLLRLNLRKMCGPF

SEQ ID NO: 3 - protein, 44 aa, Homo sapiens
KTFEKKRGKNDLQLFVMSDTTYKIYWTVILLNPCGNLHLKTTSL

SEQ ID NO: 4 - protein, 33 aa, Homo sapiens
MENSHPPTTTTSSPRRSPALRARGGTTIGEVTS

SEQ ID NO: 5 - protein, 98 aa, Homo sapiens
MSYFPILFFFSSKGVRATQSHRISQVSQNSSSWDSQRIQNCSRSSLGCSCPCTWSRCWGTCSSSWLSALTPTSTPPCTSSSPTCPWLTSVSPPPRSPR

SEQ ID NO: 6 - protein, 47 aa, Homo sapiens
PQRKRRGVPPSPPLALGPRMQLCTQLARFFPITPPVWHILGPQRHTP

SEQ ID NO: 7 - protein, 39 aa, Homo sapiens
RSNSKKKGRRNRIPAVLRTEGEPLHTPSVGMRETTGLGC

SEQ ID NO: 8 - protein, 48 aa, Homo sapiens
SSSSKTFEKKGEKNDLQLFVMSDTTYKIYWTVILLNPCGNLHLKTTSL

SEQ ID NO: 9 - protein, 76 aa, Homo sapiens
KKELEAAQKKNLLCVKCSTCPTYVKGSPSCPLRDLQTLWPILALISMSSIWGTMFSCCRLSLVQSSSWPTVLHLGH

SEQ ID NO: 10 - protein, 18 aa, Homo sapiens
AKPSSFFCRCRREYRVTM

SEQ ID NO: 11 - protein, 69 aa, Homo sapiens
DGMSTKKMCSSLALPTGLTSLILPSSDLAVLISSSTSHFLMRSPVLPSSRLTCASPQLPRMWTWSSWLK

SEQ ID NO: 12 - protein, 25 aa, Homo sapiens
EHIEALTKKRESTLGNFWMKLLQLP

SEQ ID NO: 13 - protein, 37 aa, Homo sapiens
EQVKHFFFHESSLFKLPGFLLLLVTISIFILYVIFEK

SEQ ID NO: 14 - protein, 40 aa, Homo sapiens
ESIAKIGKKNIRKLIWTKQRSFLSLFPKLRATEQMTNVGC

SEQ ID NO: 15 - protein, 53 aa, Homo sapiens
FHHPLGDTPQPSLPGPCASLLSTLSQPPPQAPSQVWTAATLRCPAVPAAACPP

SEQ ID NO: 16 - protein, 24 aa, Homo sapiens
FNPIEVMFFLSMFYLLWLNNFSSV

SEQ ID NO: 17 - protein, 57 aa, Homo sapiens
GEFLYKSKKTLNWKREPRLSYLKTMYSSLFWSQFPFLQCCHHHHLHHHYHHVLNKYI

SEQ ID NO: 18 - protein, 22 aa, Homo sapiens
INYCQKKLMLLRLNLRKMCGPF

SEQ ID NO: 19 - protein, 39 aa, Homo sapiens
KAKNSKKRGPRRKVLMVLWLPANQSLQKSQVFQWVLRTE

SEQ ID NO: 20 - protein, 49 aa, Homo sapiens
KDGEIFFWDEKTVRSNVMANVLTLNLCNRLLKSFSKWSLVQLHGIIRKN

SEQ ID NO: 21 - protein, 28 aa, Homo sapiens
KGLLSEMKKKGELSLEPWIPYLHQQKTQ

SEQ ID NO: 22 - protein, 34 aa, Homo sapiens
KKKPLKKNLHLCYYHSQSNRNKSRQMESLGMKLQ

SEQ ID NO: 23 - protein, 31 aa, Homo sapiens
KQNRPFFLPVYRQTHWRLYPKPFAGLFPLKP

SEQ ID NO: 24 - protein, 44 aa, Homo sapiens; identical to SEQ ID NO: 3

SEQ ID NO: 25 - protein, 34 aa, Homo sapiens
KTVPQKKCTNLSVPMMLTILIWKRVFILLLSDKK

SEQ ID NO: 26 - protein, 33 aa, Homo sapiens
LLVDVVYIFLTLSCLGIFPDGHIYFDFYDLLFC

SEQ ID NO: 27 - protein, 33 aa, Homo sapiens; identical to SEQ ID NO: 4

SEQ ID NO: 28 - protein, 13 aa, Homo sapiens
MIWIVFFLAPYFP

SEQ ID NO: 29 - protein, 13 aa, Homo sapiens
MQEVVVHKKRGLF

SEQ ID NO: 30 - protein, 40 aa, Homo sapiens
NKENVRDKKRATFLLALWECSLPQARLCLIVSRTLLLVQS

SEQ ID NO: 31 - protein, 35 aa, Homo sapiens
NMQNRQKKKGKNSPCCQKKLRVQNQGHLLMILLHN

SEQ ID NO: 32 - protein, 64 aa, Homo sapiens
PGQKGKKKQWSSVTSLEWTAQERGCSSWLMKQTWMKSWSLRDPSYRSILEYVSTRVLWMPTSTV

SEQ ID NO: 33 - protein, 47 aa, Homo sapiens; identical to SEQ ID NO: 6

SEQ ID NO: 34 - protein, 106 aa, Homo sapiens
QLCDNTCPLFFPPLVEKLMEPEHPEMRGEEPSTTKWSGGGGTRSTTGSSSFRKSFQTVTQTTARRERVKEGSCPRPAITSGSCARPTSACRRPSKRPSGCRWTTSS

SEQ ID NO: 35 - protein, 60 aa, Homo sapiens
QTTVEKKALRSMPKSRNQVLFRRNLTPSQLRTLAPPYMLLPQSLSQSLSRKQIPSQSMLV

SEQ ID NO: 36 - protein, 60 aa, Homo sapiens
RFQAEGSLKKTSRILNLQVLKKILRSFMKLYHSLVMCLRLRTKLEKALSALFIWPQHSYK

SEQ ID NO: 37 - protein, 45 aa, Homo sapiens
RIEVLKDDFFPLILVREWILYFVFNLHSKNRISVLLSCKVRKSYL

SEQ ID NO: 38 - protein, 77 aa, Homo sapiens
RREVSSFFFSKQGLTLLPRAGYSGTIIAHCNLELLGSRDPPTSASQSARITGMSHHTQPLPSGLRHSCNSFSRLTLL

SEQ ID NO: 39 - protein, 24 aa, Homo sapiens
SSLDIKKILFHVRNIVYGIQVMLC

SEQ ID NO: 40 - protein, 48 aa, Homo sapiens; identical to SEQ ID NO: 8

SEQ ID NO: 41 - protein, 42 aa, Homo sapiens
STFFLFVFFLGEKPQLTIVYLDRHGLLSVLLCFSNLDSFFKA

SEQ ID NO: 42 - protein, 52 aa, Homo sapiens
STPLTIGEKTEIQLTMNDSKHKLESPALKQVSPASPPTQQPQTPQDSRQVLA

SEQ ID NO: 43 - protein, 23 aa, Homo sapiens
VLGHYNNFFLPLTFSTLLWDSRH

SEQ ID NO: 44 - protein, 32 aa, Homo sapiens
VSVEPKKRNKKTKLWFSLINIHHRKNPLLPMR

SEQ ID NO: 45 - protein, 178 aa, Homo sapiens
WVNLRRGYPRLKTFGVPLGSILCLAGSLSTMAPTPPSTPMIISTTRQECGRRASVPCRPMIGSARPGPWRTSAMPSAMGVALPTSCESGRSPPATGGRMPPSGSQAPPGSQSIMMSWMPPLAPCAACPCSPAPTLCPAHPARAPTAVPAFTPLSAHPVPVLSGCHLAVRTSMLTLLPM

SEQ ID NO: 46 - protein, 13 aa, Homo sapiens
YMFLVSVIFFVCF

SEQ ID NO: 47 - protein, 28 aa, Homo sapiens
AKISFFFALCGFWQICHIKKHFQTHKLL

SEQ ID NO: 48 - protein, 18 aa, Homo sapiens
AKPSSFFCRCRREYRVTM

SEQ ID NO: 49 - protein, 87 aa, Homo sapiens
DFHLYGSYPPARQPSRPTGRTPTTRLMAPVGSVMSCSLLTQPSPASACTMPPLLHPQWAPHSAGLSPGACRPACTCISMTTISRATW

SEQ ID NO: 50 - protein, 37 aa, Homo sapiens; identical to SEQ ID NO: 13

SEQ ID NO: 51 - protein, 53 aa, Homo sapiens; identical to SEQ ID NO: 15

SEQ ID NO: 52 - protein, 24 aa, Homo sapiens
FNPIEVMFFLSMFYLLWLNNFSSV

SEQ ID NO: 53 - protein, 57 aa, Homo sapiens; identical to SEQ ID NO: 17

SEQ ID NO: 54 - protein, 65 aa, Homo sapiens
HHPMYFFLAMLSPSLTSLPAPPLYPMHSASSGSVSKKLTSMLAWPRCSLFMGSQVWSLGCSCSWL

SEQ ID NO: 55 - protein, 22 aa, Homo sapiens
INYCQKKLMLLRLNLRKMCGPF

SEQ ID NO: 56 - protein, 39 aa, Homo sapiens; identical to SEQ ID NO: 19

SEQ ID NO: 57 - protein, 32 aa, Homo sapiens
KDNHKKKQLRCWNTWAKMFFMVFLIIWQNTMF

SEQ ID NO: 58 - protein, 28 aa, Homo sapiens
KGLLSEMKKKGELSLEPWIPYLHQQKTQ

SEQ ID NO: 59 - protein, 44 aa, Homo sapiens; identical to SEQ ID NO: 3

SEQ ID NO: 60 - protein, 40 aa, Homo sapiens; identical to SEQ ID NO: 30

SEQ ID NO: 61 - protein, 35 aa, Homo sapiens; identical to SEQ ID NO: 31

SEQ ID NO: 62 - protein, 47 aa, Homo sapiens; identical to SEQ ID NO: 6

SEQ ID NO: 63 - protein, 106 aa, Homo sapiens; identical to SEQ ID NO: 34

SEQ ID NO: 64 - protein, 60 aa, Homo sapiens; identical to SEQ ID NO: 36

SEQ ID NO: 65 - protein, 77 aa, Homo sapiens; identical to SEQ ID NO: 38

SEQ ID NO: 66 - protein, 49 aa, Homo sapiens
SASNGTPLQAHPQVPALAPQAWWPARRGPLTSAPSAQQSLTKSSSSTTT

SEQ ID NO: 67 - protein, 48 aa, Homo sapiens; identical to SEQ ID NO: 8

SEQ ID NO: 68 - protein, 53 aa, Homo sapiens
VFSKKKKKKKKQHGCKGETGGSVKCGPEGAKHHAVGCPVQMGCQLLFPADPKK

SEQ ID NO: 69 - protein, 32 aa, Homo sapiens; identical to SEQ ID NO: 44

SEQ ID NO: 70 - protein, 15 aa, Homo sapiens
AKISFFFALCGFWQI

SEQ ID NO: 71 - protein, 15 aa, Homo sapiens
FFFALCGFWQICHIK

SEQ ID NO: 72 - protein, 15 aa, Homo sapiens
LCGFWQICHIKKHFQ

SEQ ID NO: 73 - protein, 15 aa, Homo sapiens
FWQICHIKKHFQTHK

SEQ ID NO: 74 - protein, 15 aa, Homo sapiens
QICHIKKHFQTHKLL

SEQ ID NO: 75 - protein, 15 aa, Homo sapiens
INYCQKKLMLLRLNL

SEQ ID NO: 76 - protein, 15 aa, Homo sapiens
QKKLMLLRLNLRKMC

SEQ ID NO: 77 - protein, 15 aa, Homo sapiens
LMLLRLNLRKMCGPF

SEQ ID NO: 78 - protein, 15 aa, Homo sapiens
KKELEAAQKKNLLCV

SEQ ID NO: 79 - protein, 15 aa, Homo sapiens
EAAQKKNLLCVKCST

SEQ ID NO: 80 - protein, 15 aa, Homo sapiens
KKNLLCVKCSTCPTY

SEQ ID NO: 81 - protein, 15 aa, Homo sapiens
LCVKCSTCPTYVKGS

SEQ ID NO: 82 - protein, 15 aa, Homo sapiens
CSTCPTYVKGSPSCP

SEQ ID NO: 83 - protein, 15 aa, Homo sapiens
PTYVKGSPSCPLRDL

SEQ ID NO: 84 - protein, 15 aa, Homo sapiens
KGSPSCPLRDLQTLW

SEQ ID NO: 85 - protein, 15 aa, Homo sapiens
SCPLRDLQTLWPILA

SEQ ID NO: 86 - protein, 15 aa, Homo sapiens
RDLQTLWPILALISM

SEQ ID NO: 87 - protein, 15 aa, Homo sapiens
TLWPILALISMSSIW

SEQ ID NO: 88 - protein, 15 aa, Homo sapiens
ILALISMSSIWGTMF

SEQ ID NO: 89 - protein, 15 aa, Homo sapiens
ISMSSIWGTMFSCCR

SEQ ID NO: 90 - protein, 15 aa, Homo sapiens
SIWGTMFSCCRLSLV

SEQ ID NO: 91 - protein, 15 aa, Homo sapiens
TMFSCCRLSLVQSSS

SEQ ID NO: 92 - protein, 15 aa, Homo sapiens
SCCRLSLVQSSSWPT

SEQ ID NO: 93 - protein, 15 aa, Homo sapiens
RLSLVQSSSWPTVLH

SEQ ID NO: 94 - protein, 15 aa, Homo sapiens
LVQSSSWPTVLHLGH

SEQ ID NO: 95 - protein, 15 aa, Homo sapiens
KTFEKKRGKNDLQLF

SEQ ID NO: 96 - protein, 15 aa, Homo sapiens
KKRGKNDLQLFVMSD

SEQ ID NO: 97 - protein, 15 aa, Homo sapiens
KNDLQLFVMSDTTYK

SEQ ID NO: 98 - protein, 15 aa, Homo sapiens
QLFVMSDTTYKIYWT

SEQ ID NO: 99 - protein, 15 aa, Homo sapiens
MSDTTYKIYWTVILL

SEQ ID NO: 100 - protein, 15 aa, Homo sapiens
TYKIYWTVILLNPCG

SEQ ID NO: 101 - protein, 15 aa, Homo sapiens
IYWTVILLNPCGNLH

SEQ ID NO: 102 - protein, 15 aa, Homo sapiens
TVILLNPCGNLHLKT

SEQ ID NO: 103 - protein, 15 aa, Homo sapiens
LLNPCGNLHLKTTSL

SEQ ID NO: 104 - protein, 15 aa, Homo sapiens
MENSHPPTTTTSSPR

SEQ ID NO: 105 - protein, 15 aa, Homo sapiens
HPPTTTTSSPRRSPA

SEQ ID NO: 106 - protein, 15 aa, Homo sapiens
TTTSSPRRSPALRAR

SEQ ID NO: 107 - protein, 15 aa, Homo sapiens
SPRRSPALRARGGTT

SEQ ID NO: 108 - protein, 15 aa, Homo sapiens
RSPALRARGGTTIGE

SEQ ID NO: 109 - protein, 15 aa, Homo sapiens
ALRARGGTTIGEVTS

SEQ ID NO: 110 - protein, 15 aa, Homo sapiens
MSYFPILFFFSSKGV

SEQ ID NO: 111 - protein, 15 aa, Homo sapiens
PILFFFSSKGVRATQ

SEQ ID NO: 112 - protein, 15 aa, Homo sapiens
FFSSKGVRATQSHRI

SEQ ID NO: 113 - protein, 15 aa, Homo sapiens
KGVRATQSHRISQVS

SEQ ID NO: 114 - protein, 15 aa, Homo sapiens
ATQSHRISQVSQNSS

SEQ ID NO: 115 - protein, 15 aa, Homo sapiens
HRISQVSQNSSSWDS

SEQ ID NO: 116 - protein, 15 aa, Homo sapiens
QVSQNSSSWDSQRIQ

SEQ ID NO: 117 - protein, 15 aa, Homo sapiens
NSSSWDSQRIQNCSR

SEQ ID NO: 118 - protein, 15 aa, Homo sapiens
WDSQRIQNCSRSSLG

SEQ ID NO: 119 - protein, 15 aa, Homo sapiens
RIQNCSRSSLGCSCP

SEQ ID NO: 120 - protein, 15 aa, Homo sapiens
CSRSSLGCSCPCTWS

SEQ ID NO: 121 - protein, 15 aa, Homo sapiens
SLGCSCPCTWSRCWG

SEQ ID NO: 122 - protein, 15 aa, Homo sapiens
SCPCTWSRCWGTCSS

SEQ ID NO: 123 - protein, 15 aa, Homo sapiens
TWSRCWGTCSSSWLS

SEQ ID NO: 124 - protein, 15 aa, Homo sapiens
CWGTCSSSWLSALTP

SEQ ID NO: 125 - protein, 15 aa, Homo sapiens
CSSSWLSALTPTSTP

SEQ ID NO: 126 - protein, 15 aa, Homo sapiens
WLSALTPTSTPPCTS

SEQ ID NO: 127 - protein, 15 aa, Homo sapiens
LTPTSTPPCTSSSPT

SEQ ID NO: 128 - protein, 15 aa, Homo sapiens
STPPCTSSSPTCPWL

SEQ ID NO: 129 - protein, 15 aa, Homo sapiens
CTSSSPTCPWLTSVS

SEQ ID NO: 130 - protein, 15 aa, Homo sapiens
SPTCPWLTSVSPPPR

SEQ ID NO: 131 - protein, 15 aa, Homo sapiens
CPWLTSVSPPPRSPR

SEQ ID NO: 132 - protein, 15 aa, Homo sapiens
PQRKRRGVPPSPPLA

SEQ ID NO: 133 - protein, 15 aa, Homo sapiens
RRGVPPSPPLALGPR

SEQ ID NO: 134 - protein, 15 aa, Homo sapiens
PPSPPLALGPRMQLC

SEQ ID NO: 135 - protein, 15 aa, Homo sapiens
PLALGPRMQLCTQLA

SEQ ID NO: 136 - protein, 15 aa, Homo sapiens
GPRMQLCTQLARFFP

SEQ ID NO: 137 - protein, 15 aa, Homo sapiens
QLCTQLARFFPITPP

SEQ ID NO: 138 - protein, 15 aa, Homo sapiens
QLARFFPITPPVWHI

SEQ ID NO: 139 - protein, 15 aa, Homo sapiens
FFPITPPVWHILGPQ

SEQ ID NO: 140 - protein, 15 aa, Homo sapiens
TPPVWHILGPQRHTP

SEQ ID NO: 141 - protein, 15 aa, Homo sapiens
RSNSKKKGRRNRIPA

SEQ ID NO: 142 - protein, 15 aa, Homo sapiens
KKKGRRNRIPAVLRT

SEQ ID NO: 143 - protein, 15 aa, Homo sapiens
RRNRIPAVLRTEGEP

SEQ ID NO: 144 - protein, 15 aa, Homo sapiens
IPAVLRTEGEPLHTP

SEQ ID NO: 145 - protein, 15 aa, Homo sapiens
LRTEGEPLHTPSVGM

SEQ ID NO: 146 - protein, 15 aa, Homo sapiens
GEPLHTPSVGMRETT

SEQ ID NO: 147 - protein, 15 aa, Homo sapiens
HTPSVGMRETTGLGC

SEQ ID NO: 148 - protein, 15 aa, Homo sapiens
SSSSKTFEKKGEKND

SEQ ID NO: 149 - protein, 15 aa, Homo sapiens
KTFEKKGEKNDLQLF

SEQ ID NO: 150 - protein, 15 aa, Homo sapiens
KKGEKNDLQLFVMSD

SEQ ID NO: 151 - protein, 11 aa, Homo sapiens
KQNRPFFLPVY

SEQ ID NO: 152 - protein, 10 aa, Homo sapiens
YPKPFAGLFP

SEQ ID NO: 153 - protein, 9 aa, Homo sapiens
KQNRPFFLP

SEQ ID NO: 154 - protein, 9 aa, Homo sapiens
QNRPFFLPV

SEQ ID NO: 155 - protein, 9 aa, Homo sapiens
NRPFFLPVY

SEQ ID NO: 156 - protein, 9 aa, Homo sapiens
YPKPFAGLF

SEQ ID NO: 157 - protein, 10 aa, Homo sapiens
SLEPWIPYLH

SEQ ID NO: 158 - protein, 9 aa, Homo sapiens
SLEPWIPYL

SEQ ID NO: 159 - protein, 9 aa, Homo sapiens
LEPWIPYLH

SEQ ID NO: 160 - protein, 10 aa, Homo sapiens
WMKSWSLRDP

SEQ ID NO: 161 - protein, 11 aa, Homo sapiens
LCLAGSLSTMA

SEQ ID NO: 162 - protein, 9 aa, Homo sapiens
CLAGSLSTM

SEQ ID NO: 163 - protein, 16 aa, Homo sapiens
MENSHPPTTTTSSPRR

SEQ ID NO: 164 - protein, 16 aa, Homo sapiens
CTNLSVPMMLTILIWK

SEQ ID NO: 165 - protein, 15 aa, Homo sapiens
NLLCVKCSTCPTYVK

SEQ ID NO: 166 - protein, 20 aa, Homo sapiens
IPAVLRTEGEPLHTPSVGMR

SEQ ID NO: 167 - protein, 36 aa, Homo sapiens
GETGGSVKCGPEGAKHHAVGCPVQMGCQLLFPADPK

SEQ ID NO: 168 - protein, 38 aa, Homo sapiens
ASVPCRPMIGSARPGPWRTSAMPSAMGVALPTSCESGR

SEQ ID NO: 169 - protein, 20 aa, Homo sapiens
IPAVLRTEGEPLHTPSVGMR

SEQ ID NO: 170 - protein, 14 aa, Homo sapiens
TKLWFSLINIHHRK

SEQ ID NO: 171 - protein, 17 aa, Homo sapiens
KLRVQNQGHLLMILLHN

(19) United States
(12) Patent Application Publication
(10) Pub. No.: US 2021/0393752 A1
(43) Pub. Date: Dec. 23, 2021

(54) PEPTIDES, COMPOSITIONS AND VACCINES FOR TREATMENT OF MICROSATELLITE INSTABLITY HYPERMUTATED TUMORS AND METHODS OF USE THEREOF

(71) Applicant: Icahn School of Medicine at Mount Sinai, New York, NY (US)

(72) Inventors: Nina Bhardwaj, West Orange, NJ (US); Vladimir Roudko, New York, NY (US); Cansu Cimen Bozkus, New York, NY (US)

(21) Appl. No.: 17/290,128

(22) PCT Filed: Nov. 6, 2019
(86) PCT No.: PCT/US19/60040

§ 371 Date: Apr. 29, 2021

Related U.S. Application Data
(60) Provisional application No. 62/813,829, filed on Mar. 5, 2019.
(60) Provisional application No. 62/756,305, filed on Nov. 6, 2018.

(51) Int. Cl. A61K 39/00; A61K 35/17; A61P 35/00; A61P 37/04

(52) U.S. Cl. CPC A61K 39/0011; A61P 37/04; A61P 35/00; A61K 35/17

(57) ABSTRACT

Neoantigenic peptides useful for the treatment of MSI-H tumors, vaccines and composition comprising the peptides, and methods of inducing or enhancing an immune response and of treating MSI-H tumors are provided.

### CROSS REFERENCE TO RELATED APPLICATION

This application claims priority of U.S. Provisional Application No. 62/756,305 filed Nov. 6, 2018, and U.S. Provisional Application No. 62/813,829 filed Mar. 5, 2019, the disclosure of which are incorporated herein by reference in their entireties.

### FIELD OF THE INVENTION

The invention relates generally to the fields of medicine, oncology and molecular biology. In particular, the invention relates to peptides, compositions and vaccines for generating an immune response and treating cancer in an individual having a microsatellite instability hypermutated (MSI-H) tumor or at risk of developing such a tumor.

### BACKGROUND

Cancer-specific neoantigens, resulting from genetic alterations accumulated by tumor cells, encode novel stretches of amino acids that are not present in the normal genome. These tumor-specific peptides therefore have not been negatively selected by the immune system as “self” proteome. Thus, total neoantigen load inferred through in silico analysis of whole-exome sequencing data from patients' tumors can be used as a predictor of positive responses to immunotherapy regiments as well as used for peptide-based vaccine design (Luksza, M. et al. Nature 551, 517-520 (2017); Balachandran, V. P. et al. Nature 551, S12-S16 (2017); Charoentong, P. et al. Cell Reports 18, 248-262 (2017); Hugo, W. et al. Cell 165, 35-44 (2016)). Neoantigen vaccines are developed by comparing the genotype of tumor cells with patient's matching normal tissue or blood. Collected somatic missense and frameshift mutations are then converted to corresponding tumor-specific peptides, which then are screened for MHC-I epitopes through running either experimental functional tests or in silico prediction algorithms. Several algorithms exist, optimizing prediction of epitope-HLA interactions in silico, making it possible to predict MHC class I, and to a lesser extent, MEW class II tumor neoepitopes. Alternatively, mass-spectrometry based approaches to predict tumor epitopes now exist as well. Subsequently, these epitopes can be used for short and long peptide-based vaccines, boosting dendritic-cell (DC) based vaccinations, priming adoptive autologous T cell transfer, and gene-modified cell therapies (Branca, M. A. Nat. Biotechnol. 34, 1019-1024 (2016)).

Personalized vaccines using neoantigens that arise from tumor-specific genomic alterations are currently in evaluation in multiple clinical trials. However, these tumor antigens are highly personalized and require patient-specific sequencing approaches for their identification.

MSI-H tumors have high mutation rates due to dysfunction of a specific DNA damage response pathway. Approximately 20% of endometrial cancer tumors and 10% of colorectal cancer (CRC) tumors and stomach cancer tumors are MSI-H. MSI-H tumors have been shown to respond well to PD-1/PD-L1 inhibitors, and this treatment is now approved in the second line setting. Due to the presence of high loads of tumor-specific antigens, and strong effector T cell infiltration, MSI-H tumors emerge as an important model system for neoantigen-based immunotherapy in therapeutic and protective settings and to improve upon the use of checkpoint inhibitors, which still do not cure the majority of patients. However, current peptide-based cancer vaccination strategies utilize a personalized approach requiring costly sequencing techniques to identify patient-specific tumor associated antigens, which then can be formulated into a vaccine. Current peptide-based cancer vaccination strategies also require lengthy amounts of time. It may take more than a month from tumor sample collection to the actionable peptide mix. In light of these drawbacks, there is an unmet meet for a common (i.e., universal) vaccine design, which can be used for immunization of a large number of cancer patients with minimal time spent in diagnostics.

### SUMMARY OF THE INVENTION

In one embodiment, the present disclosure provides neoantigenic tumor-specific peptides, wherein the tumor is an MSI-H tumor. In some embodiments, the MSI-H tumor is an endometrial, colorectal, or stomach tumor.

In another embodiment, the present disclosure provides nucleic acids encoding the neoantigenic tumor-specific peptides, and vectors comprising the nucleic acids.

In another embodiment, the present disclosure provides a vaccine for the treatment of a MSI-H tumor. In some embodiments, the tumor is an endometrial, colorectal, or stomach tumor. The vaccine comprises one or more neoantigenic tumor-specific peptides or nucleic acids encoding such peptides, wherein the tumor is a MSI-H tumor. The vaccine may further comprise an adjuvant.

In another embodiment, the present disclosure provides a composition comprising one or more neoantigenic tumor-specific peptides, wherein the tumor is a MSI-H tumor, and a pharmaceutically acceptable carrier. In some embodiments, the tumor is a MSI-H endometrial, colorectal, or stomach tumor.

In another embodiment, the present disclosure provides a method of inducing or enhancing an immune response to a MSI-H tumor in a subject. The method includes administering to the subject a vaccine comprising one or more neoantigenic tumor-specific peptides, or a composition comprising one or more neoantigenic tumor-specific peptides, wherein the tumor is a MSI-H tumor, in a therapeutically effective amount to induce an immune response in the subject. In an embodiment of the method, the subject has MSI-H endometrial cancer, MSI-H colorectal cancer, or MSI-H stomach cancer. In another embodiment, the subject is at risk of developing a MSI-H cancer, including for example a MSI-H endometrial cancer, MSI-H colorectal cancer, or MSI-H stomach cancer.

In another embodiment, the present disclosure provides a method for the treatment of a MSI-H tumor comprising administering a vaccine of the invention to a subject in need of such treatment. The method may further comprise administering a second anti-cancer agent to the subject, wherein the second anti-cancer agent is administered simultaneously or sequentially. In an embodiment of the method, the subject has MSI-H endometrial cancer, MSI-H colorectal cancer, or MSI-H stomach cancer. In another embodiment, the subject is at risk of developing a MSI-H cancer, including for example a MSI-H endometrial cancer, MSI-H colorectal cancer, or MSI-H stomach cancer.

In one embodiment, the present disclosure provides a pharmaceutical composition for use in adoptive cell therapy to treat a tumor in a patient in need thereof comprising a population of T-cells expressing one or more chimeric antigen receptors (CARs) or one or more T-cell receptors (TCRs) that are reactive to at least one neoantigenic peptide having the amino acid sequence of one of SEQ ID NOs:1-69 or a fragment thereof. In embodiments, the pharmaceutical composition comprises a population of T-cells that have been engineered to express one or more CARs or one or more TCRs that are reactive to at least one neoantigenic peptide having the amino acid sequence of one of SEQ ID NOs:1-69 or a fragment thereof. In embodiments, the at least one neoantigenic peptide has the amino acid sequence of one of SEQ ID NOs: 2, 3, 6, 8, and 47 or a fragment thereof. In embodiments, (i) the tumor is a MSI-H endometrial tumor, and the at least one neoantigenic peptide has the amino acid sequence of one of SEQ ID NOs:1-9 or a fragment thereof; (ii) the tumor is a MSI-H colorectal tumor, and the at least one neoantigenic peptide has the amino acid sequence of one of SEQ ID NOs:10-46 or a fragment thereof; and (iii) the tumor is a MSI-H stomach tumor, and the at least one neoantigenic peptide has the amino acid sequence of one of SEQ ID NOs:47-69 or a fragment thereof. The present disclosure further provides a method of inducing or enhancing an immune response in a subject having a MSI-H tumor or at risk of having an MSI-H tumor, comprising administering to the subject a therapeutically effective amount of the pharmaceutical composition comprising a population of T-cells expressing one or more CARs or one or more TCRs that are reactive to at least one neoantigenic peptides having the amino acid sequence of one of SEQ ID NOs:1-69 or a fragment thereof.

### BRIEF DESCRIPTION OF THE DRAWINGS

FIG. 1 shows the overlapping peptide design for each neoantigenic peptide from shared frameshift peptides of MSI-H uterine corpus endometrial cancer (UCEC) patients. Peptide 1 has the sequence of SEQ ID NO:1. Peptides 1.1 to 1.5 have the sequences of SEQ ID Nos: 70-74, respectively. Peptide 2 has the sequence of SEQ ID NO:2. Peptides 2.1 to 2.3 have the sequences of SEQ ID NOs: 75-77, respectively. Peptide 9 has the sequence of SEQ ID NO:9. Peptides 3.1 to 3.17 have the sequences of SEQ ID NOs: 78-94, respectively. Peptide 3 has the sequence of SEQ ID NO:3. Peptides 4.1 to 4.9 have the sequences of SEQ ID NOs:95-103, respectively. Peptide 4 has the sequence of SEQ ID NO: 4. Peptides 5.1 to 5.6 have the sequences of SEQ ID NOs:104-109, respectively. Peptide 5 has the sequence of SEQ ID NO:5. Peptides 6.1 to 6.22 have the sequences of SEQ ID NOs:110-131, respectively. Peptide 6 has the sequence of SEQ ID NO:6. Peptides 7.1 to 7.9 have the sequences of SEQ ID NOs:132-140, respectively. Peptide 7 has the sequence of SEQ ID NO:7. Peptides 8.1 to 8.7 have the sequences of SEQ ID NOs: 141-147, respectively. Peptide 8 has the sequence of SEQ ID NO:8. Peptides 9.1 to 9.3 have the sequences of SEQ ID NOs: 148-150, respectively.

FIG. 2 shows that shared frameshift-peptides predicted from UCEC MSI-H patients elicit 688 T cell responses. FIG. 2A shows an overview of T cell immunogenicity assay used to evaluate antigen-specific T cell responses. PBMCs from healthy donors (HD) were expanded in vitro following stimulation with pools of overlapping long peptides (OLPs: 15 amino acid (aa) long, overlapping with an offset of 4 aa) spanning each frameshift-peptide. Expanded T cells (5×104 cells/well) were re-stimulated with either the peptide pool they were expanded with or the control peptide pool MOG. FIG. 2B shows representative IFN-γ ELISPOT images for HD13 and FIG. 2C shows images for selected responsive HD. FIG. 2D shows a summary of ELISPOT data (n=14). Statistical significance for MOG vs OLPs was evaluated by Wilcoxon signed-rank test. FIG. 2E shows representative flow cytometry plots. Summary of data (n=15) is shown for IFN-γ in CD8 (FIG. 2F) and CD4 (FIG. 2G) T cell subsets. Stimulation with CEFT was used as a control (FIG. 2H). Statistical significance for DMSO vs OLPs was evaluated by Wilcoxon signed-rank test. **p=0.0032 for SLC22A9 and **0.0031 for CEFT. H. Frequency of IFN-γ or TNF-α producing CD8+ T cells upon stimulation with WT OLP pool. CEFT and PMA/Ionomycin stimulation was used as a control. The spot numbers and % IFN-γ values were calculated by subtracting the values obtained after MOG or DMSO stimulation from the values after OLP pool stimulation and negative values were set to zero.

FIG. 3 shows data from MHC-I epitopes predicted from frameshift peptides that are presented by HCT116 cell line and derived from colon cancer with MSI-H genotype. FIG. 3A shows the schema of the predicted frameshift peptide of SEQ ID NO:23. Epitopes eluted from MHC-I and identified by MS/MS are KQNRPFFLPVY (SEQ ID NO; 151) and YPKPFAGLFP (SEQ ID NO:152). The position of the frameshift mutation within the peptide sequence is amino acids 8 and 9 (LP). FIG. 3B shows MS/MS spectra of MHC-I epitopes by PepQuery. Statistical significance of the peptide-spectrum match (PSM) is defined by p-value. FIG. 3C shows reference and alternative allele frequencies in HCT116 cell line as estimated by WES and/or RNAseq experiments derived from Cancer Cell Line Encyclopedia (CCLE). FIG. 3D shows frequency of 9-mer epitope presentation in MSI-H patient cohorts, Tumor Cancer Genome Atlas (TCGA) dataset. 9-mer epitopes are derived from antigens identified in MS/MS spectra. Peptide KQNRPFFLP has the sequence of SEQ ID NO:153. Peptide QNRPFFLPV has the sequence of SEQ ID NO: 154. Peptide NRPFFLPVY has the sequence of SEQ ID NO: 155. Peptide YPKPFAGLF has the sequence of SEQ ID NO: 156.

FIG. 4 shows data from MHC-I epitopes predicted from frameshift peptides that are presented by HCT116 cell line and derived from colon cancer with MSI-H genotype. FIG. 4A shows the schema of the predicted frameshift peptide of SEQ ID NO:21. An epitope eluted from MHC-I and identified by MS/MS is SLEPWIPYLH (SEQ ID NO:157). The position of the frameshift mutation within the peptide sequence is amino acids 8 and 9 (KK). FIG. 4B shows MS/MS spectra of MHC-I epitopes by PepQuery. Statistical significance of the peptide-spectrum match (PSM) is defined by p-value. FIG. 4C shows reference and alternative allele frequencies in HCT116 cell line as estimated by WES and/or RNAseq experiments derived from Cancer Cell Line Encyclopedia (CCLE). FIG. 4D shows frequency of 9-mer epitope presentation in MSI-H patient cohorts, Tumor Cancer Genome Atlas (TCGA) dataset. 9-mer epitopes are derived from antigens identified in MS/MS spectra. Peptide SLEPWIPYL has the sequence of SEQ ID NO:158. Peptide LEPWIPYLH has the sequence of SEQ ID NO: 159.

FIG. 5 shows data from MHC-I epitopes predicted from frameshift peptides that are presented by HCT116 cell line and derived from colon cancer with MSI-H genotype. FIG. 5A shows the schema of the predicted frameshift peptide of SEQ ID NO:32. An epitope eluted from MHC-I and identified by MS/MS is WMKSWSLRDP (SEQ ID NO:160). The position of the frameshift mutation within the peptide sequence is amino acids 8 and 9 (KQ). FIG. 5B shows MS/MS spectra of MHC-I epitopes by PepQuery. Statistical significance of the peptide-spectrum match (PSM) is defined by p-value. FIG. 5C shows reference and alternative allele frequencies in HCT116 cell line as estimated by WES and/or RNAseq experiments derived from Cancer Cell Line Encyclopedia (CCLE).

FIG. 6 shows data from MHC-I epitopes predicted from frameshift peptides that are presented by HCT116 cell line and derived from colon cancer with MSI-H genotype. FIG. 6A shows the schema of the predicted frameshift peptide of SEQ ID NO:45. An epitope eluted from MHC-I and identified by MS/MS is LCLAGSLSTMA (SEQ ID NO:161). The position of the frameshift mutation within peptide sequence is amino acids 8 and 9 (YP). FIG. 4B shows MS/MS spectra of MHC-I epitopes by PepQuery. Statistical significance of the peptide-spectrum match (PSM) is defined by p-value. FIG. 4C shows reference and alternative allele frequencies in HCT116 cell line as estimated by WES and/or RNAseq experiments derived from Cancer Cell Line Encyclopedia (CCLE). FIG. 4D shows frequency of 9-mer epitope presentation in MSI-H patient cohorts, Tumor Cancer Genome Atlas (TCGA) dataset. 9-mer epitopes are derived from antigens identified in MS/MS spectra. Peptide CLAGSLS™ has the sequence of SEQ ID NO: 162.

FIG. 7 shows MS/MS spectra of predicted frameshift peptides in whole-cell MS/MS experiment from TCGA-AA-AOOR COAD MSI-H tumor sample, Clinical Proteomic Tumor Analysis Consortium dataset (CPTAC). Predicted frameshift peptides for SEQ ID NOs: 27, 25, and 29 are represented in schema. The tryptic peptide for SEQ ID NO:27 is MENSHPPTTTTSSPRR (SEQ ID NO: 163) and the frameshift mutation is at amino acid positions 8 and 9. The tryptic peptide for SEQ ID NO:25 is CTNLSVPMMLTILIWK (SEQ ID NO: 164) and the frameshift mutation is at amino acid positions 8 and 9. The tryptic peptide for SEQ ID NO:9 is NLLCVKCSTCPTYVK (SEQ ID NO: 165) and the frameshift mutation is at amino acid positions 8 and 9. Statistical significance of peptide-spectrum match by PepQuery: p-value and hyperscore, are shown under the peptide schema. PSM MS/MS spectra identified by PepQuery for each represented peptide is shown on the right.

FIG. 8 shows Top scored PSM spectra of tryptic peptides matched to predicted frameshift peptides from prospectively collected colon (FIG. 8A) and endometrial (FIG. 8B) tumor samples, whole cell MS/MS CPTAC datasets. FIG. 8C is a table with Hyperscores and p-values of PSM spectra, PepQuery. The frameshift peptides in the table have SEQ ID NOs: 7, 68, 45, 7, 44, and 31. Tryptic peptide IPAVLRTEGEPLHTPSVGMR has the sequence of SEQ ID NO: 166. Tryptic peptide GETGGSVKCGPEGAKHHAVGCPVQMGCQLLFPADPK ha the sequence of SEQ ID NO: 167. Tryptic peptide ASVPCRPMIGSARPGPWRTSAMPSAMGVALPTSCESGR has the sequence of SEQ ID NO: 168. Tryptic peptide IPAVLRTEGEPLHTPSVGMR has the sequence of SEQ ID NO: 169. Tryptic peptide TKLWFSLINIHHRK has the sequence of SEQ ID NO: 170. Tryptic peptide KLRVQNQGHLLMILLHN has the sequence of SEQ ID NO: 171.

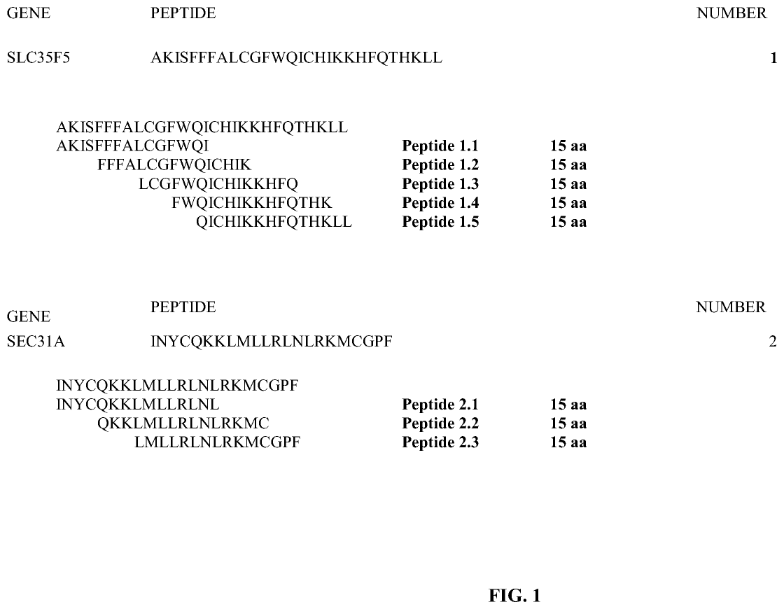

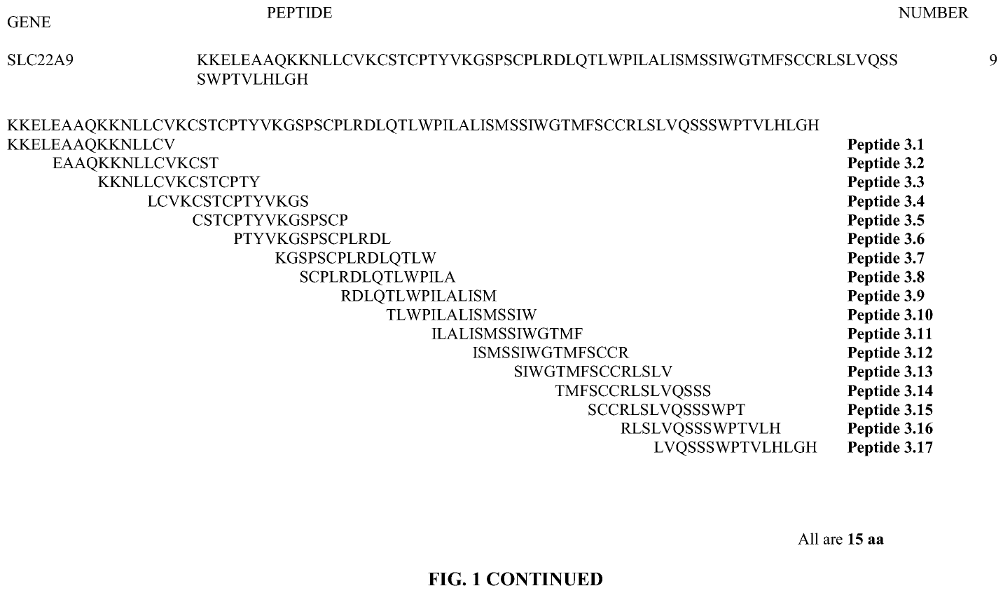

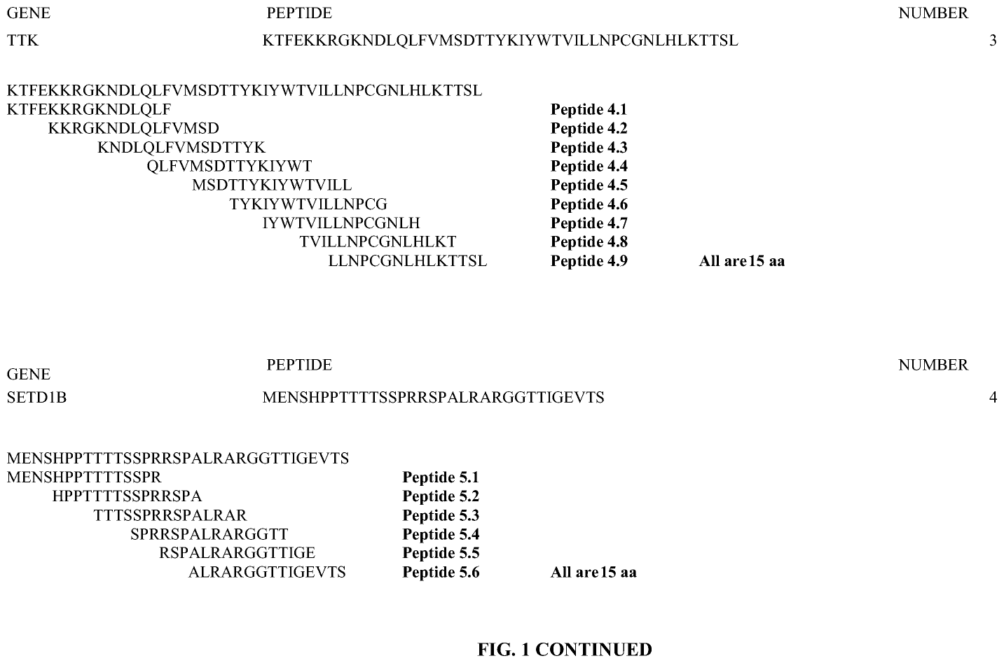

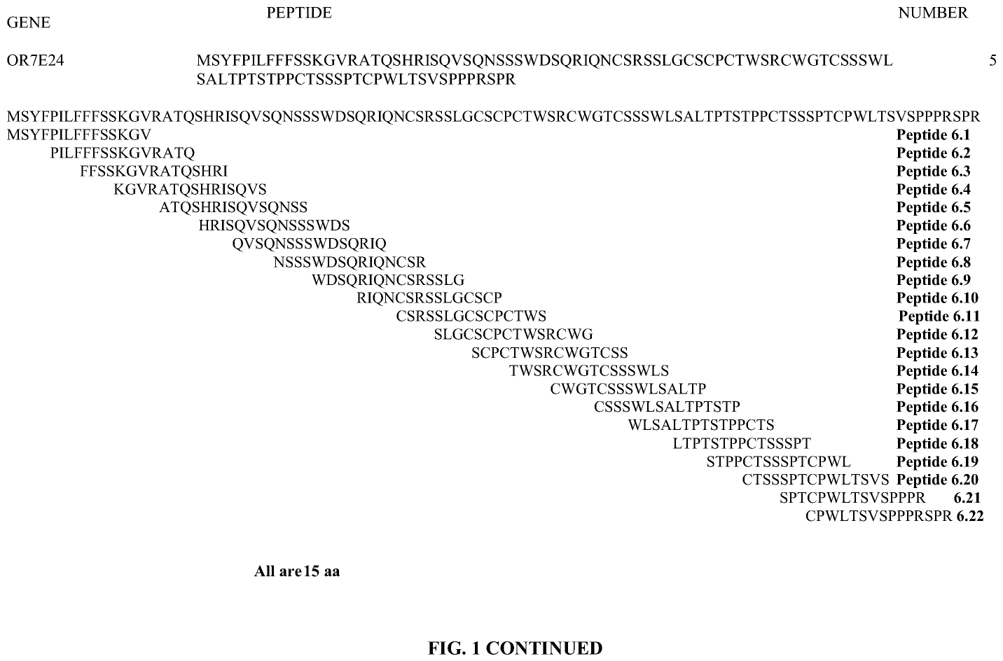

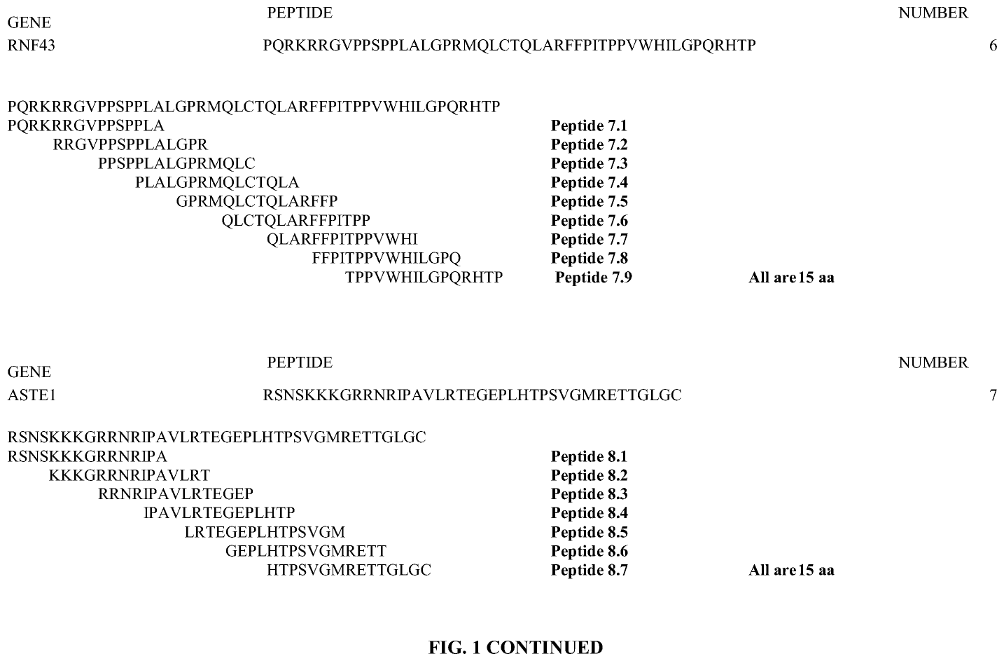

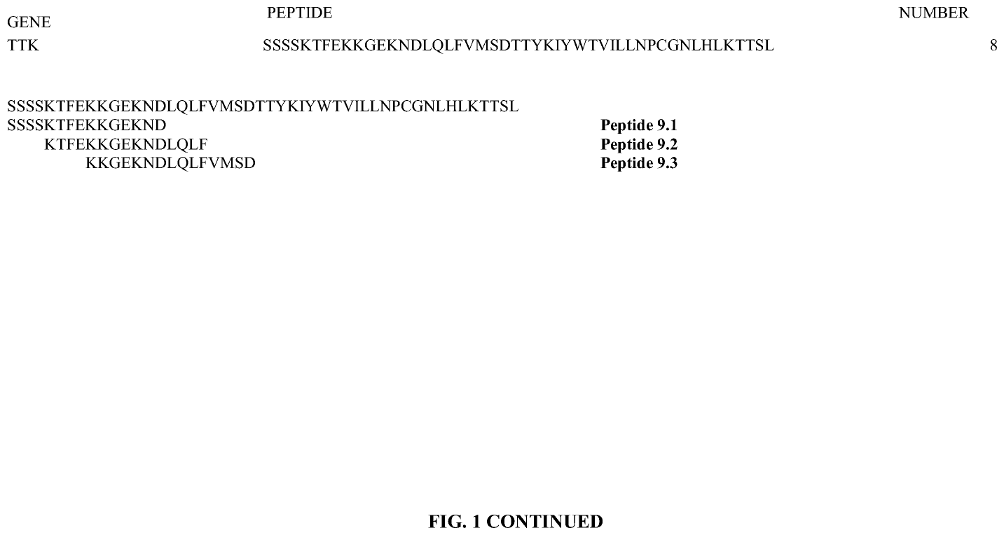

### DETAILED DESCRIPTION OF THE INVENTION

Microsatellite instability is a hypermutation pattern caused by defects in the mismatch repair system. Microsatellite instability has been described in several types of cancer, including endometrial, colorectal, and stomach cancers. Other cancers identified as MSI-H that can be treated in accordance with the methods of the present invention include adrenocortical carcinoma, breast carcinoma, bladder carcinoma, esophageal carcinoma, head and neck squamous cell carcinoma, kidney carcinoma, lower grade glioma, liver hepatocellular carcinoma, mesothelioma, ovarian cancer, prostate adenocarcinoma, rectal adenocarcinoma, skin cutaneous carcinoma, and uterine carcinoma (Bonneville, R. et al. JCO Precision Oncology 1, 1-15 (2017)).

In one embodiment, described herein are neoantigenic peptides that are useful for vaccines in MSI-H cancer subjects and in subjects at risk for developing MSI-H tumors. Nine endometrial MSI-H cancer peptides, 37 MSI-H colorectal cancer (CRC) peptides, and 23 MSI-H stomach cancer neoantigenic peptides have been identified. Approximately 90% of MSI-H endometrial patients have at least one of these neoantigens, and the vast majority have several of these neoantigens. Five of these neoantigens are also present in greater than 25% of MSI-H CRC and stomach cancer tumors. The endometrial cancer neoantigenic peptides are useful for vaccines for treatment of MSI-H endometrial tumors. The colorectal cancer neoantigenic peptides are useful for vaccines for treatment of MSI-H colorectal tumors. The stomach cancer neoantigenic peptides are useful for vaccines for treatment of MSI-H stomach tumors. The neoantigenic peptides that are common to more than one MSI-H cancer can be used as universal vaccines for MSI-H tumors.

Accordingly, described herein is a neoantigenic peptide that is present in an MSI-H tumor and that is immunogenic in a subject having the MSI-H tumor. In one embodiment, the neoantigenic peptide is present in a MSI-H endometrial tumor and is immunogenic in a subject having a MSI-H endometrial tumor. In some embodiments, the peptide has an amino acid sequence comprising one of the following amino acid sequences:

(SEQ ID NO: 1)
AKISFFFALCGFWQICHIKKHFQTHKLL;
(SEQ ID NO: 2)
INYCQKKLMLLRLNLRKMCGPF;
(SEQ ID NO: 3)
KTFEKKRGKNDLQLFVMSDTTYKIYWTVILLNPCGNLHLKTTSL;
(SEQ ID NO: 4)
MENSHPPTTTTSSPRRSPALRARGGTTIGEVTS;
(SEQ ID NO: 5)
MSYFPILFFFSSKGVRATQSHRISQVSQNSSSWDSQRIQNCSRSSLGCSC
PCTWSRCWGTCSSSWLSALTPTSTPPCTSSSPTCPWLTSVSPPPRSPR;

(SEQ ID NO: 6)
PQRKRRGVPPSPPLALGPRIVIQLCTQLARFFPITPPVWHILGPQRHTP
(SEQ ID NO: 7)
RSNSKKKGRRNRIPAVLRTEGEPLHTPSVGMRETTGLGC;
(SEQ ID NO: 8)
SSSSKTFEKKGEKNDLQLFVMSDTTYKIYWTVILLNPCGNLHLKTTSL;
(SEQ ID NO: 9)
KKELEAAQKKNLLCVKCSTCPTYVKGSPSCPLRDLQTLWPILALISMSSI
WGTIVIFSCCRLSLVQSSSWPTVLHLGH.

In another embodiment, the neoantigenic peptide is present in a MSI-H colorectal tumor and is immunogenic in a subject having a colorectal MSI-H tumor. In some embodiments, the peptide has an amino acid sequence comprising one of the following amino acid sequences:

(SEQ ID NO: 10)
AKPSSFFCRCRREYRVTM;
(SEQ ID NO: 11)
DGMSTKKMCSSLALPTGLTSLILPSSDLAVLISSSTSHFLMRSPVLPSSR
LTCASPQLPRMWTWSSWLK;
(SEQ ID NO: 12)
EHIEALTKKRESTLGNFWMKLLQLP;
(SEQ ID NO: 13)
EQVKHFFFHESSLFKLPGFLLLLVTISIFILYVIFEK;
(SEQ ID NO: 14)
ESIAKIGKKNIRKLIWTKQRSFLSLFPKLRATEQMTNVGC;
(SEQ ID NO: 15)
FHHPLGDTPQPSLPGPCASLLSTLSQPPPQAPSQVWTAATLRCPAVPAAA
CPP;
(SEQ ID NO: 16)
FNPIEVMFFLSMFYLLWLNNFSSV;
(SEQ ID NO: 17)
GEFLYKSKKTLNWKREPRLSYLKTMYSSLFWSQFPFLQCCHHHHLHHHYH
HVLNKYI;
(SEQ ID NO: 18)
INYCQKKLMLLRLNLRKMCGPF;
(SEQ ID NO: 19)
KAKNSKKRGPRRKVLMVLWLPANQSLQKSQVFQWVLRTE;
(SEQ ID NO: 20)
KDGEIFFWDEKTVRSNVMANVLTLNLCNRLLKSFSKWSLVQLHGIIRKN;
(SEQ ID NO: 21)
KGLLSEMKKKGELSLEPWIPYLHQQKTQ;
(SEQ ID NO: 22)
KKKPLKKNLHLCYYHSQSNRNKSRQMESLGMKLQ;
(SEQ ID NO: 23)
KQNRPFFLPVYRQTHWRLYPKPFAGLFPLKP;
(SEQ ID NO: 24)
KTFEKKRGKNDLQLFVMSDTTYKIYWTVILLNPCGNLHLKTTSL;
(SEQ ID NO: 25)
KTVPQKKCTNLSVPMMLTILIWKRVFILLLSDKK;
(SEQ ID NO: 26)
LLVDVVYIFLTLSCLGIFPDGHIYFDFYDLLFC;
(SEQ ID NO: 27)
MENSHPPTTTTSSPRRSPALRARGGTTIGEVTS;
(SEQ ID NO: 28)
MIWIVFFLAPYFP;
(SEQ ID NO: 29)
MQEVVVHKKRGLF;
(SEQ ID NO: 30)
NKENVRDKKRATFLLALWECSLPQARLCLIVSRTLLLVQS;
(SEQ ID NO: 31)
NMQNRQKKKGKNSPCCQKKLRVQNQGHLLMILLHN;
(SEQ ID NO: 32)
PGQKGKKKQWSSVTSLEWTAQERGCSSWLMKQTWMKSWSLRDPSYRSILE
YVSTRVLWMPTSTV;
(SEQ ID NO: 33)
PQRKRRGVPPSPPLALGPRIVIQLCTQLARFFPITPPVWHILGPQRHTP;
(SEQ ID NO: 34)
QLCDNTCPLFFPPLVEKLMEPEHPEMRGEEPSTTKWSGGGGTRSTTGSSS
FRKSFQTVTQTTARRERVKEGSCPRPAITSGSCARPTSACRRPSKRPSGC
RWTTSS;
(SEQ ID NO: 35)
QTTVEKKALRSMPKSRNQVLFRRNLTPSQLRTLAPPYMLLPQSLSQSLSR
KQIPSQSMLV;
(SEQ ID NO: 36)
RFQAEGSLKKTSRILNLQVLKKILRSFMKLYHSLVMCLRLRTKLEKALSA
LFIWPQHSYK;
(SEQ ID NO: 37)
RIEVLKDDFFPLILVREWILYFVFNLHSKNRISVLLSCKVRKSYL;
(SEQ ID NO: 38)
RREVSSFFFSKQGLTLLPRAGYSGTIIAHCNLELLGSRDPPTSASQSARI
TGMSHHTQPLPSGLRHSCNSFSRLTLL;
(SEQ ID NO: 39)
SSLDIKKILFHVRNIVYGIQVMLC;
(SEQ ID NO: 40)
SSSSKTFEKKGEKNDLQLFVMSDTTYKIYWTVILLNPCGNLHLKTTSL;
(SEQ ID NO: 41)
STFFLFVFFLGEKPQLTIVYLDRHGLLSVLLCFSNLDSFFKA;
(SEQ ID NO: 42)
STPLTIGEKTEIQLTMNDSKHKLESPALKQVSPASPPTQQPQTPQDSRQV
LA;
(SEQ ID NO: 43)
VLGHYNNFFLPLTFSTLLWDSRH;
(SEQ ID NO: 44)
VSVEPKKRNKKTKLWFSLINIHHRKNPLLPMR;
(SEQ ID NO: 45)
WVNLRRGYPRLKTFGVPLGSILCLAGSLSTMAPTPPSTPMIISTTRQECG
RRASVPCRPMIGSARPGPWRTSAMPSAMGVALPTSCESGRSPPATGGRMP
PSGSQAPPGSQSIMMSWMPPLAPCAACPCSPAPTLCPAHPARAPTAVPAF
TPLSAHPVPVLSGCHLAVRTSMLTLLPM;
(SEQ ID NO: 46)
YMFLVSVIFFVCF.

In another embodiment, the neoantigenic peptide is present in a MSI-H stomach tumor and is immunogenic in a subject having a MSI-H stomach tumor. In some embodiments, the peptide has an amino acid sequence comprising one of the following amino acid sequences:

(SEQ ID NO: 47)
AKISFFFALCGFWQICHIKKHFQTHKLL;
(SEQ ID NO: 48)
AKPSSFFCRCRREYRVTM;
(SEQ ID NO: 49)
DFHLYGSYPPARQPSRPTGRTPTTRLMAPVGSVMSCSLLTQPSPASACTM
PPLLHPQWAPHSAGLSPGACRPACTCISMTTISRATW;
(SEQ ID NO: 50)
EQVKHFFFHESSLFKLPGFLLLLVTISIFILYVIFEK;
(SEQ ID NO: 51)
FHHPLGDTPQPSLPGPCASLLSTLSQPPPQAPSQVWTAATLRCPAVPAAA
CPP;
(SEQ ID NO: 52)
FNPIEVMFFLSMFYLLWLNNFSSV;
(SEQ ID NO: 53)
GEFLYKSKKTLNWKREPRLSYLKTMYSSLFWSQFPFLQCCHHHHLHHHYH
HVLNKYI;
(SEQ ID NO: 54)
HHPMYFFLAMLSPSLTSLPAPPLYPMHSASSGSVSKKLTSMLAWPRCSLF
MGSQVWSLGCSCSWL;
(SEQ ID NO: 55)
INYCQKKLMLLRLNLRKMCGPF;
(SEQ ID NO: 56)
KAKNSKKRGPRRKVLMVLWLPANQSLQKSQVFQWVLRTE;
(SEQ ID NO: 57)
KDNHKKKQLRCWNTWAKMFFMVFLIIWQNTMF;
(SEQ ID NO: 58)
KGLLSEMKKKGELSLEPWIPYLHQQKTQ;
(SEQ ID NO: 59)
KTFEKKRGKNDLQLFVMSDTTYKIYWTVILLNPCGNLHLKTTSL;
(SEQ ID NO: 60)
NKENVRDKKRATFLLALWECSLPQARLCLIVSRTLLLVQS;
(SEQ ID NO: 61)
NMQNRQKKKGKNSPCCQKKLRVQNQGHLLMILLHN;
(SEQ ID NO: 62)
PQRKRRGVPPSPPLALGPRMQLCTQLARFFPITPPVWHILGPQRHTP;
(SEQ ID NO: 63)
QLCDNTCPLFFPPLVEKLMEPEHPEMRGEEPSTTKWSGGGGTRSTTGSSS
FRKSFQTVTQTTARRERVKEGSCPRPAITSGSCARPTSACRRPSKRPSGC
RWTTSS;
(SEQ ID NO: 64)
RFQAEGSLKKTSRILNLQVLKKILRSFMKLYHSLVMCLRLRTKLEKALSA
LFIWPQHSYK;
(SEQ ID NO: 65)
RREVSSFFFSKQGLTLLPRAGYSGTIIAHCNLELLGSRDPPTSASQSARI
TGMSHHTQPLPSGLRHSCNSFSRLTLL;
(SEQ ID NO: 66)
SASNGTPLQAHPQVPALAPQAWWPARRGPLTSAPSAQQSLTKSSSSTTT;
(SEQ ID NO: 67)
SSSSKTFEKKGEKNDLQLFVMSDTTYKIYWTVILLNPCGNLHLKTTSL;
(SEQ ID NO: 68)
VFSKKKKKKKKQHGCKGETGGSVKCGPEGAKHHAVGCPVQMGCQLLFPAD
PKK;
(SEQ ID NO: 69)
VSVEPKKRNKKTKLWFSLINIHHRKNPLLPMR.

In another embodiment, the neoantigenic peptide is present in more than one type of MSI-H tumor type and is immunogenic in a subject having an MSI-H tumor. In this embodiment, the peptide has an amino acid sequence comprising one of the following amino acid sequences:

(SEQ ID NO: 2)
INYCQKKLMLLRLNLRKMCGPF;
(SEQ ID NO: 3)
KTFEKKRGKNDLQLFVMSDTTYKIYWTVILLNPCGNLHLKTTSL;
(SEQ ID NO: 6)
PQRKRRGVPPSPPLALGPRMQLCTQLARFFPITPPVWHILGPQRHTP;
(SEQ ID NO: 8)
SSSSKTFEKKGEKNDLQLFVMSDTTYKIYWTVILLNPCGNLHLKTTSL;
(SEQ ID NO: 47)
AKISFFFALCGFWQICHIKKHFQTHKLL.

In another embodiment, the neoantigenic peptide has an amino acid sequence comprising a contiguous fragment of the sequence of any of the sequences of SEQ ID NOs: 1-69, wherein the fragment is capable of eliciting an immune response. In a preferred embodiment, the fragment comprises at least 8, at least 9, at least 10, at least 11, at least 12, at least 13, at least 14, at least 15, at least 16, at least 17, at least 18, at least 19, or at least 20 contiguous amino acids of any of the sequences of SEQ ID NOs: 1-69.

In another embodiment, the neoantigenic peptide is a variant of one of the neoantigenic peptides described above. The variant may include amino acid deletions, substitutions, or additions, so long as it remains capable of eliciting an immune response.

In another embodiment, the present disclosure provides nucleic acids encoding the neoantigenic peptides described above, and vectors comprising the nucleic acids. In some embodiments, the vector is an expression vector. One of ordinary skill in the art can utilize well-known methods of recombinant technology to make such nucleic acids and vectors.

Also described herein is a tumor vaccine comprising at least one neoantigenic peptide as described hereinabove. In the tumor vaccine, the at least one neoantigen peptide may be a plurality (e.g., 2, 3, 4, 5, 6, 7, 8, 9, 10, 15, 20, etc.) of neoantigenic peptides. The tumor vaccine may further include an adjuvant and/or a pharmaceutically acceptable carrier. Adjuvants are known in the art. Various adjuvants that can be used to further increase the immunological response depend on the host species and include Freund's adjuvant (complete and incomplete), cytokines, growth factors, mineral gels such as aluminum hydroxide, surface-active substances such as lysolecithin, pluronic polyols, polyanions, peptides, oil emulsions, keyhole limpet hemocyanin, and dinitrophenol.

In one preferred embodiment, the vaccine comprises one or more neoantigenic peptides that are common to more than one MSI-H cancer type, i.e., peptides having SEQ ID NOs: 2, 3, 6, 8, or 47 or fragments or variants thereof as defined above. This universal vaccine is useful for treating MSI-H tumors of multiple types. In another preferred embodiment, the vaccine comprises all of the peptides having SEQ ID NOs: 2, 3, 6, 8, and 47, or fragments and variants of each of those peptides.

Further described herein is a composition including at least one neoantigenic peptide as described above, in a therapeutically effective amount to induce an immune response in a subject, and a pharmaceutically acceptable carrier. In the composition, the at least one neoantigenic peptide may be a plurality (e.g., 2, 3, 4, 5, 6, 7, 8, 9, 10, 15, 20, etc.) of neoantigenic peptides. A typical composition for inducing and enhancing an immune response and for treating cancer in a subject having an MSI-H tumor typically includes a therapeutically effective amount (i.e., an amount effective to induce an immune response) of at least one neoantigenic peptide that is present in an MSI-H tumor. Such a composition may be in a form suitable for administration either by itself or alternatively, using a delivery vehicle (e.g., liposomes, micelles, nanospheres, etc.) Any suitable delivery vehicles and techniques for delivering peptides to cells may be used.

In another embodiment, the present invention provides a method of inducing or enhancing an immune response to a MSI-H tumor in a subject. The method includes administering to the subject a therapeutically effective amount of a tumor vaccine including at least one neoantigenic peptide as described above that is present in a MSI-H tumor and that is immunogenic in a subject having the MSI-H tumor, or a composition including at least one neoantigenic peptide as described above that is present in a MSI-H tumor in a therapeutically effective amount to induce an immune response in a subject and a pharmaceutically acceptable carrier. In an embodiment of this method, the subject has MSI-H endometrial cancer and the vaccine or composition comprises at least one neoantigenic peptide selected from SEQ ID NOs: 1-9 or a fragment or variant thereof as defined hereinabove. In another embodiment of this method, the subject has MSI-H colorectal cancer and the vaccine or composition comprises at least one neoantigenic peptide selected from SEQ ID NOs: 10-46 or a fragment or variant thereof as defined hereinabove. In another embodiment of this method, the subject has MSI-H stomach cancer and the vaccine or composition comprises at least one neoantigenic peptide selected from SEQ ID NOs: 47-69 or a fragment or variant thereof as defined hereinabove.

In another embodiment of this method, the subject has MSI-H cancer and the vaccine or composition comprises at least one neoantigenic peptide selected from SEQ ID NOs: 2, 3, 6, 8, and 47 or a fragment or variant thereof as defined hereinabove.

Typically, the therapeutically effective amount induces a CD8⁺ T-cell response to the neoantigen in the subject. It can also induce a CD4⁺ T-cell response that is also an effective anti-tumor response which can also assist in the CD8⁺ T-cell response. Administration of the tumor vaccine or the composition to the subject can reduce or eliminate metastatic spread of cancer cells in the subject. In an embodiment, the subject has a MSI-H tumor and administration of the tumor vaccine or the composition to the subject reduces tumor growth rate in the subject. In the methods, a vaccine or a composition including at least one neoantigenic peptide that is present in an MSI-H tumor and that is immunogenic in a subject (e.g., a subject having an MSI-H tumor) may be administered at a same or different time point as administration to the subject of a second anti-cancer agent. In an embodiment, both a composition including a neoantigenic peptide as described herein and a second composition including a second anti-cancer agent are administered. A third anti-cancer agent may also be administered. The methods can be used to treat MSI-H cancer (e.g., MSI-H endometrial cancer, MSI-H colorectal, MSI-H stomach cancer, etc.) in a subject in need thereof. In some embodiments, the subject (e.g., human) in need thereof has a cancer that is characterized by one or more MSI-H tumors. In some embodiments, these methods can also be used to induce preventive memory responses in a high-risk subject, e.g., a subject having Lynch syndrome.

In another embodiment, the present disclosure provides a method for the treatment of a MSI-H tumor comprising administering a vaccine or composition as described above to a subject in need of such treatment. In an embodiment of the method, the subject has MSI-H endometrial cancer, MSI-H colorectal cancer, or MSI-H stomach cancer. In another embodiment, the subject is at risk of developing a MSI-H cancer, including for example a MSI-H endometrial cancer, MSI-H colorectal cancer, or MSI-H stomach cancer. The method may further comprise administering an second anti-cancer agent to the subject, wherein the anti-cancer agent is administered simultaneously or sequentially. Anti-cancer agents include, e.g., anti-neoplastic agents, anti-tumor agents, anti-angiogenic agents, and immunotherapeutic agents. A list of such other anti-cancer agents is included in U.S. Patent Application Publication No. US 2017/0151240, which is incorporated herein by reference in its entirety.

Any suitable methods of administering a neoantigenic peptide as described herein, or compositions or vaccines containing the neoantigenic peptides, to a subject may be used. In these methods, the peptides, compositions and vaccines may be administered to a subject by any suitable route, e.g., oral, buccal (e.g., sub-lingual), intratumoral, parenteral (e.g., subcutaneous, intramuscular, intradermal, or intravenous), topical (i.e., both skin and mucosal surfaces, including airway surfaces), rectal, vaginal, and transdermal administration. In an embodiment, the peptides, compositions and vaccines may be administered systemically by intravenous injection or parenterally by subcutaneous injection. In another embodiment, the peptides, compositions and vaccines may be administered directly to a target site, by, for example, surgical delivery to an internal or external target site, or by catheter to a site accessible by a blood vessel. If administered via intravenous injection, the peptides, compositions and vaccines may be administered in a single bolus, multiple injections, or by continuous infusion (e.g., intravenously, by peritoneal dialysis, pump infusion). For parenteral administration, the peptide, composition or vaccine is preferably formulated in a sterilized pyrogen-free form.

Neoantigenic peptides, vaccines and compositions described herein for enhancing and inducing immune responses, for treating cancer, and for inducing preventive memory responses in a high-risk subject can be administered as a monotherapy or as part of a combination therapy with any other anti-cancer agent in the methods described herein. In some embodiments of combination therapy, a therapeutic vaccine or composition contains both a neoantigenic peptide (a first anti-cancer agent) as described herein and a second anti-cancer agent. In other embodiments of a combination therapy, a first composition may include a neoantigenic peptide as described herein, and a second composition may include the second anti-cancer agent. In such embodiments, the first composition may be administered at the same time point or approximately the same time point as the second composition. Alternatively, the first and second compositions may be administered at different time points. A neoantigenic peptide as described herein can be used in a combination therapy that includes one or more of immunotherapy, chemotherapy, radiotherapy, and surgery.

As indicated above, a neoantigenic peptide as described herein, or composition or vaccine containing the neoantigenic peptide, may be in a form suitable for sterile injection. To prepare such a composition, the suitable active therapeutic agent(s) (i.e., a therapeutically effective amount of a neoantigenic peptide as described herein) is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution (D5W, 0.9% sterile saline). The aqueous formulation may also contain one or more preservatives (e.g., methyl, ethyl or n-propyl p-hydroxybenzoate). In cases where the therapeutic agent(s) is only sparingly or slightly soluble in water, a dissolution enhancing or solubilizing agent can be added, or the solvent may include 10-60% w/w of propylene glycol or the like. The neoantigenic peptides as described herein, or compositions or vaccines containing the neoantigenic peptides, may be administered to an individual (e.g., rodents, humans, nonhuman primates, canines, felines, ovines, bovines) in any suitable formulation according to conventional pharmaceutical practice (see, e.g., Remington: The Science and Practice of Pharmacy (21 st ed.), ed. A. R. Gennaro, Lippincott Williams & Wilkins, (2005) and Encyclopedia of Pharmaceutical Technology, (3^{rd} ed.) eds. J. Swarbrick and J. C. Boylan, Marcel Dekker, CRC Press, New York (2006), a standard text in this field, and in USP/NF). A description of exemplary pharmaceutically acceptable carriers and diluents, as well as pharmaceutical formulations, can be found in Remington, supra. Other substances may be added to the compositions to stabilize and/or preserve them.

The therapeutic methods described herein in generally include administration of a therapeutically effective amount of one or more neoantigenic peptides as described herein, or composition or vaccine containing the neoantigenic peptides, to a subject in need thereof, particularly a human. Such treatment will be suitably administered to individuals, particularly humans, suffering from, having, susceptible to, or at risk for a disease, disorder, or symptom thereof (e.g., cancer characterized by MSI-H tumors, Lynch syndrome). Determination of those subjects or individuals “at risk” can be made by any objective or subjective determination by a diagnostic test or opinion of a subject or health care provider. For example, numerous prognostic markers or factors for categorizing colorectal cancer patients or individuals for likely outcome of treatment are known. See, e.g., Lin P S & Semrad T J, Methods Mol Biol. 2018 1765:281-297; and Zacharakis et al., Anticancer Res, 2010 30(2): 653-660. In some embodiments, the individual in need of treatment is afflicted with a relapsed endometrial, colorectal, or stomach cancer.

The neoantigenic peptides as described herein, or compositions or vaccines containing the neoantigenic peptides, are preferably administered to a subject in need thereof (e.g., human having cancer characterized by MSI-H tumors) in an effective amount, that is, an amount capable of producing a desirable result in a treated individual. Desirable results include one or more of, for example, inducing or enhancing an immune response, reducing tumor size, reducing cancer cell metastasis, and prolonging survival. Such a therapeutically effective amount can be determined according to standard methods. Toxicity and therapeutic efficacy of the neoantigenic peptides as described herein, or compositions or vaccines containing the neoantigenic peptides, that are utilized in the methods described herein can be determined by standard pharmaceutical procedures. As is well known in the medical and veterinary arts, dosage for any one individual depends on many factors, including the individual's size, body surface area, age, the particular composition to be administered, time and route of administration, general health, and other drugs being administered concurrently. A delivery dose of a composition as described herein is determined based on preclinical efficacy and safety.

In another embodiment, described herein are kits for inducing or enhancing an immune response and for treating cancer (e.g., MSI-H endometrial cancer, MSI-H colorectal cncer, MSI-H stomach cancer, etc.) in a subject. A typical kit includes a composition including a pharmaceutically acceptable carrier (e.g., a physiological buffer) and a therapeutically effective amount of at least one neoantigenic peptide as described herein, or composition or vaccine containing the neoantigenic peptide; and instructions for use. A kit can also include a second anti-cancer agent. Kits also typically include a container and packaging. Instructional materials for preparation and use of the peptides, vaccines and compositions described herein are generally included. While the instructional materials typically include written or printed materials, they are not limited to such. Any medium capable of storing such instructions and communicating them to an end user is encompassed by the kits herein. Such media include, but are not limited to electronic storage media, optical media, and the like. Such media may include addresses to internet sites that provide such instructional materials.

In another embodiment, the present invention provides adoptive cell therapy (ACT) methods that utilize one or more of the neoantigenic peptides described herein to create a population of T cells that are reactive to a MSI-H tumor. Adoptive cell therapy is a form of immunotherapy that involves the transfer of immune cells with antitumor activity into patients.

In embodiments, the adoptive cell therapy involves isolating lymphocytes and genetically engineering the lymphocytes to express antitumor T cell receptors (TCRs) or chimeric antigen receptors (CARs) before expanding the lymphocytes and administering them to a patient in need thereof. The lymphocytes used for infusion can be isolated from the patient (autologous cell therapy) or from a donor (allogeneic cell therapy). In an embodiment of this method, autologous or allogenic T cells are engineered to express a TCR or CAR that specifically recognizes a neoantigen described herein.

In embodiments of the invention, the adoptive cell therapy comprises administration of a therapeutically effective amount of a T cell composition to a patient in need thereof wherein the T cell composition comprises a population of T cells that express a CAR or a TCR that is reactive to one or more neoantigenic peptides selected from SEQ ID NOs: 2, 3, 6, 8, and 47 or a fragment or variant thereof as defined hereinabove. In an embodiment of this method, the subject has MSI-H endometrial cancer and the T cell composition comprises a population of T cells that express a CAR or a TCR that is reactive to at least one neoantigenic peptide selected from SEQ ID NOs: 1-9 or a fragment or variant thereof as defined hereinabove. In another embodiment of this method, the subject has MSI-H colorectal cancer and the T cell composition comprises a population of T cells that express a CAR or a TCR that is reactive to at least one neoantigenic peptide selected from SEQ ID NOs: 10-46 or a fragment or variant thereof as defined hereinabove. In another embodiment of this method, the subject has MSI-H stomach cancer and the composition comprises a population of T cells that express a CAR or a TCR that is reactive to at least one neoantigenic peptide selected from SEQ ID NOs: 47-69 or a fragment or variant thereof as defined hereinabove.

The term “chimeric antigen receptor” or “CAR” or “CARs” as used herein refers to engineered receptors, which graft antigen specificity onto a cytotoxic cell, for example T cells, NK cells and macrophages. The CARs of the invention may include at least one neoantigen specific targeting region, an extracellular spacer domain, a transmembrane domain, one or more co-stimulatory domains, and an intracellular signaling domain. In embodiments, the co-stimulatory domain(s), and/or the intracellular signaling domain are optional. In another embodiment, the CAR is a bispecific CAR, which is specific to two different antigens or epitopes. After the neoantigen specific targeting region binds specifically to a target neoantigen, the intracellular signaling domain activates intracellular signaling directing T cell specificity and reactivity toward a selected neoantigen target in a non-MHC-restricted manner. The non-MHC-restricted antigen recognition gives the T cells expressing the CAR the ability to recognize an antigen independent of antigen processing, thus bypassing a major mechanism of tumor escape.

The neoantigen specific targeting region of the CAR comprises an antibody, especially a single-chain antibody, or a fragment thereof. The neoantigen specific targeting region may include a full length heavy chain, an Fab fragment, a single chain Fv (scFv) fragment, a divalent single chain antibody or a diabody, each of which being specific to a target neoantigen described herein.

The extracellular spacer domain of the CAR is located between the neoantigen specific targeting region and the transmembrane domain and may be an optional component for the CAR. The extracellular spacer domain may include a domain selected from hinge regions of antibodies, Fc fragments of antibodies, CH2 regions of antibodies, CH3 regions of antibodies, artificial spacer sequences or combinations thereof. Examples of extracellular spacer domains include CD8a hinge, polypeptides spacers which may be as small as, three glycines (Gly), as well as CH1 and CH3 domains of IgGs.

The transmembrane domain of the CAR is a region that is capable of spanning the plasma membrane of the cytotoxic cells. The transmembrane domain is selected from a transmembrane region of a transmembrane protein such as, for example, Type I transmembrane proteins, an artificial hydrophobic sequence or a combination thereof. Examples of the transmembrane domain include the transmembrane regions of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Synthetic transmembrane domains may include a triplet of phenylalanine, tryptophan and valine. Optionally, a short oligo- or polypeptide linker, preferably between 2 and 10 amino acids in length, may form the linkage between the transmembrane domain and the intracellular signaling domain of the CAR. A glycine-serine doublet provides a particularly suitable linker between the transmembrane domain and the intracellular signaling domain.

The intracellular signaling domains used in the CAR may include intracellular signaling domains of other immune signaling receptors, including, but not limited to, first, second, and third generation T cell signaling proteins including CD3, B7 family costimulatory, and Tumor Necrosis Factor Receptor (TNFR) superfamily receptors. Additionally intracellular signaling domains include signaling domains used by NK and NKT cells such as signaling domains of NKp30 (B7-H6) and DAP12, NKG2D, NKp44, NKp46, DAP10, and CD3z. Additionally intracellular signaling domains may include signaling domains of human immunoglobulin receptors that contain immunoreceptor tyrosine based activation motif (ITAM) such as FcgammaRI, FcgammaRIIA, FcgammaRIIC, FcgammaRIIIA, FcRL5. In some embodiments, the intracellular signaling domain includes a cytoplasmic signaling domain of CD3 gamma, CD3 zeta, CD3 delta, CD3 epsilon, TCR zeta, FcR gamma, FcR beta, CD5, CD22, CD79a, CD79b, or CD66d.

The CAR of the present invention may include one or more a co-stimulatory domains, which, e.g., enhance cell proliferation and survival. The one or more co-stimulatory domains may be selected from co-stimulatory domains of proteins in the TNFR superfamily, CD28, CD137 (4-1BB), CD134 (OX40), Dap1O, CD27, CD2, CD7, CD5, ICAM-1, LFA-1 (CD1 1a/CD18), Lck, TNFR-I, PD-1, TNFR-II, Fas, CD30, CD40, ICOS LIGHT, NKG2C, B7-H3, or combinations thereof. If the CAR includes more than one co-stimulatory domain, these domains may be arranged in tandem, optionally separated by a linker.

In embodiment, the adoptive cell therapy methods of the invention involve isolation of lymphocytes from a patient, stimulating and culturing the lymphocytes in vitro to expand the population with antitumor activity, and then infusing the lymphocytes into the patient in need thereof. Lymphocytes used for adoptive transfer can either be derived from resected tumors (e.g., tumor infiltrating lymphocytes or TILs), from the lymphatics or lymph nodes, or from the blood. In embodiments of the invention, the adoptive cell therapy comprises administration of a therapeutically effective amount of a T cell composition to a patient in need thereof wherein the T cell composition comprises a plurality of T cells reactive to one or more neoantigenic peptides selected from SEQ ID NOs: 2, 3, 6, 8, and 47 or a fragment or variant thereof as defined hereinabove. In an embodiment of this method, the subject has MSI-H endometrial cancer and the T cell composition comprises a population of T cells reactive to at least one neoantigenic peptide selected from SEQ ID NOs: 1-9 or a fragment or variant thereof as defined hereinabove. In another embodiment of this method, the subject has MSI-H colorectal cancer and the T cell composition comprises a population of T cells reactive to at least one neoantigenic peptide selected from SEQ ID NOs: 10-46 or a fragment or variant thereof as defined hereinabove. In another embodiment of this method, the subject has MSI-H stomach cancer and the composition comprises a population of T cells reactive to at least one neoantigenic peptide selected from SEQ ID NOs: 47-69 or a fragment or variant thereof as defined hereinabove. The methods in some embodiments utilize activated T cells induced by dendritic cells (DCs) loaded with one or more the neoantigenic peptides, or a fragments or variants thereof. The T cells and DCs, for example, can be derived from the patient's peripheral blood mononuclear cells (PBMCs). Multiple-antigen loaded DCs can be prepared by isolation and exposure of DCs to a plurality of the neoantigenic peptides, or a fragments or variants thereof. Activated T cells can be prepared by co-culturing a population of T cells with the antigen loaded DCs. Optionally, the population of T cells is contacted with one or more cytokines (e.g., IL-2) and optionally anti-CD3 antibody prior to and/or during the co-culturing. The activated T cells may be expanded in culture prior to administration to the patient, thereby eliciting an adoptive immune response against the one or more if the neoantigens in vivo. Optionally, the multiple-antigen loaded DCs can be administered to the individual to trigger active immunity against the one or more neoantigens. TCRs that are reactive to a neoantigenic peptide (presented by an antigen presenting cell) can be cloned from T-cells that are activated and expanded as described above.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

“Microsatellite Instable tumors,” “microsatellite instability hypermutated tumors”, “MSI-H tumors” and “MSI high tumors” as used herein are tumors having a greater than normal number of microsatellites. Microsatellite instability is a hypermutation pattern caused by defects in the mismatch repair system. Examples of MSI-H tumors include endometrial cancer, colorectal cancer, and stomach cancer. However, any tumor displaying instability in two or more of the five markers (BAT25, BAT26, D2S123, D5S346, and D17S250) as recommend by the 1997 NCI consensus meeting (Boland, C R et al, Cancer Res. 58:5248-57 (1998)) or more than 30% of markers in the marker panel defined by Hegde, M et al. Genet Med. 16:101-16 (2014) is defined as MSI-H.

As used herein the term “antigen” is a substance that induces an immune response, e.g., a CD8⁺ T cell response.

As used herein the term “immunogenic” is the ability to elicit an immune response, e.g., via T cells, B cells, or both.

By the term “neoantigenic peptide” is meant an antigenic peptide that is encoded by one or more tumor-specific mutated genes. The mutation that can give rise to a new sequence that represents a neoantigen can include a frameshift or nonframeshift indel (insertion or deletion), missense or nonsense substitution, splice site alteration, genomic rearrangement or gene fusion, or any genomic or expression alteration giving rise to a tumor-specific open reading frame (ORF). A mutation can also include a splice variant. A neoantigenic peptide is typically present in a subject's tumor cell or tissue but not in the subject's corresponding normal cell or tissue. Neoantigenic that are common to MSI-H tumors (e.g., 2, 3, 4, 5, 10, 15, etc., MSI-H tumors) are particularly useful in the compositions, vaccines and methods described herein. The term “neoantigenic peptide” as used herein is understood to include the peptides identified by sequence identification numbers as well as fragments and variants thereof as defined hereinabove.

The terms “agent” and “therapeutic agent” as used herein refer to a chemical entity or biological product, or combination of chemical entities or biological products, administered to a subject to treat a disease or condition (e.g., cancer). Examples of agents include small molecule drugs and biologics.

The terms “patient,” “subject” and “individual” are used interchangeably herein, and mean a subject to be treated, diagnosed, and/or to obtain a biological sample from. Subjects include, but are not limited to, humans, non-human primates, horses, cows, sheep, pigs, rats, mice, dogs, and cats. A human in need of cancer treatment is an example of a subject. A human who is at risk for cancer is another example of a subject.

As used herein, the terms “treatment” and “therapy” are defined as the application or administration of a therapeutic agent or therapeutic agents to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

All publications, patent applications, and patents mentioned herein are incorporated by reference in their entireties. In the case of conflict, the present specification, including definitions, will control. The particular embodiments discussed below are illustrative only and not intended to be limiting.

### EXAMPLES

The present invention is further illustrated by the following examples. The examples are provided for illustration only and should not be construed as limiting the scope of the invention in any way.

### Example 1—WIDESPREAD OCCURRENCE OF COMMON IMMUNOGENIC ANTIGENS IN TUMORS WITH MICROSATELLITE INSTABILITY

A new computational pipeline, called UniVac (Universal Vaccine), was used to identify highly frequent shared tumor epitopes. The approach was applied to MSI-H patient cohorts from The Cancer Genome Atlas (TCGA) to determine whether such shared antigens could be identified and be immunogenic. Selected epitopes identified in MSI-H endometrial cancer patients were experimentally validated in complimentary immunological assays using peripheral blood mononuclear cells (PBMCs) from healthy donors. Through these computational and experimental analyses a comprehensive landscape of immunogenic tumor-specific antigens emerged, revealing the design of a common vaccine, which is of immediate clinical relevance. By applying statistically tailored vaccines for MSI-H endometrial, colorectal and stomach carcinomas, one can achieve objective responses in existing neoplasms or develop preventive memory responses in high-risk patient populations, such as Lynch syndrome.

An approach was developed and applied to detect immunogenic, tumor-specific frameshifts, which occur with high frequency among patients and are highly clonal within each tumor itself. This computational method relies on calling frameshift-derived “novel” peptide sequences, which encode multiple MHC-I epitopes. Finally, the most common epitopes were selected for experimental verification and vaccine formulation.

### Results

### Estimation of Frameshift Mutation Load in MSI-H Colorectal, Stomach and Endometrial Patient Cohort of TCGA

The frameshift mutational load of TCGA patients was analyzed in the perspective of designing potential universal neoantigen tumor vaccines. Frameshift mutations have been analyzed in colorectal MSI-H patients previously, and insertions/deletions have been analyzed in pan-cancer scale (Turajlic, S. et al. Lancet Oncol. 18, 1009-1021 (2017); Marty, R. et al. Cell 0, 1-12 (2017); Mlecnik, B. et al. Immunity 44, 698-711 (2016)). However, it was decided to perform statistical analyses with an emphasis on potential vaccine design. TCGA has sequencing information of ˜5000 patients with diverse tumor types, which vary greatly in mutation properties. The majority of these tumors are microsatellite stable or their status is unknown. However, ˜10% of endometrial, colorectal and stomach adenocarcinomas are diagnosed as microsatellite unstable, MSI-H (UCEC, colon adenocarcinoma (COAD), stomach adenocarcinoma (STAD) respectively). Frameshift load of these three tumor types was particularly high only in a fraction of patients, and correlated well with their MSI-H status. The majority of MSI-H frameshifts are nucleotide deletions by nature.

### Colon, Stomach and Endometrial MSI-H Adenocarcinomas are Enriched in Potentially Immunogenic Frameshift Peptides

To identify potentially immunogenic frameshift mutations, a frameshift neoantigen calling pipeline was developed (see Experimental procedures below). Following this computational approach, distributions of frameshift mutations were analyzed, and frameshift peptides and MHC-I epitopes in MSI-H UCEC, COAD and STAD cohorts of TCGA patients were derived. Multiple frameshift mutations in microsatellite regions target similar genes among many patients of all three tumor types. The frequency of shared peptides and predicted epitopes, derived from same frameshift mutations, is smaller than the frequency of commonly mutated genes, with some gene candidates having same epitopes in up to 25% of patients in UCEC, and above 50% in COAD and STAD MSI-H patients. Colon and stomach MSI-H tumors had a twice higher frequency of shared events then endometrial MSI-H tumors on average. This was attributed to the differences in underlying profiles of mutated gene drivers, which may have resulted in differences in mutation frequencies.

A mutation ranking system was developed in order to confidently short-list high-frequency immunogenic frameshift mutations. The quality metrics of each frameshift mutation: “PASS” or “NO PASS”, derived from applied mutation filters, were aggregated across all MSI-H patients. That allowed the ranking of the frameshift mutations depending on their annotation frequency of being PASSed by TCGA mutation callers. Also, it was noted that the average length of MSI-H frameshift peptides is 20-30 amino acid residues, thus potentially encoding multiple immunogenic epitopes per mutation. Frameshift peptides generate 1-5 epitopes, which bind multiple HLA-alleles. The more epitopes per peptide, the more epitopes this frameshift binds. However, the relation between epitope binding and HLA-allele frequencies is non-linear: less frequent alleles tend to have similar epitope binding frequency. Using developed ranking as a measure of mutation quality, the distribution of shared frameshift peptides with population-wise frequency above 25% was plotted for each tumor type separately. Thus 9, 37 and 33 frameshift peptides were identified as candidates for vaccine design in endometrial, colorectal and stomach, respectively, MSI-H patients. All identified peptides share high mutation confident scores, and encode multiple MHC-I epitopes, which interact with the majority of HLA-types of patient cohorts. Notably, among detected peptides, 5 frameshifts were shared across all MSI-H tumor types, and are thus suitable for a common universal MSI-H vaccine.

To estimate the expression level of genes encoding frameshifted peptides, RNA expression levels derived from TCGA RNAseq samples of matched MSI-H patients were analyzed. While MSI-H patients had been ranked by the overall frameshift load, showing high-load and low-load patients, the corresponding gene expression did not follow the same trend. This supports conclusion that frameshifted genes are not turned off in tumor cells, i.e., that frameshift mutations do not alter gene expression in MSI-H tumors.

Furthermore, it was speculated whether frameshift load has a predictive power for immunotherapy outcomes within the MSI-H patient cohort. Despite the high response rate to PD-1 blockage and acceptance of MSI-H as a biomarker for PD-1 therapy (Dudley, J. C., et al. Clin. Cancer Res. 22, 813-820 (2016); Le, D. T. et al. N. Engl. J. Med. 372, 2509-2520 (2015)), genetic differences between patients may underlie the responsiveness. For this purpose, the clinical outcomes of TCGA MSI-H patients were correlated with frameshift load estimations. However, no significant differences were detected between survival curves between high- and low-frameshift load MSI-H patients of three studied tumor types. Thus, frameshift load alone may not be sufficient to ameliorate MSI-H biomarker.

### TCGA can be Used to Guide the Design of Minimal Peptide Vaccine for Endometrial MSI-H Carcinomas

Due to patient availability, vaccine designs for endometrial MSI-H tumor type were evaluated initially. MHC-I epitopes from selected 9 frameshift peptides were detected at least once in >80% of the MSI-H UCEC TCGA patient cohort, binding to almost all HLA-alleles of same patient population. A mixture of MHC-I epitopes derived from all 9 peptides can reach significantly high coverage. After analyzing population-wide distributions of these peptides, the selected frameshift frequencies within each patient's tumor were estimated. To do that, the corresponding frameshift allele frequencies in normal and tumor samples were estimated. Frameshift alleles were nearly undetectable in normal tissues, while their frequency rose up to 40% in tumors. This indicates that the 9-peptide vaccine mix targets almost all cellular content of a patient's malignancy.

Similar to the described above strategy, shared frameshift peptides of a colon and stomach MSI-H patient cohort were analyzed. Pulling all peptides together predicted epitopes in >75% of all patients. The 9 shared peptides, combined together, covered almost all most-frequent HLA-alleles. These data support the use of the neoantigens described herein in a vaccine and for vaccine design in colon and stomach MSI-H patients.

### Similarity of Selected Tumor Epitopes to Virus Antigens

The intrinsic properties of frameshift-derived MHC-I epitopes were also investigated. To do that, neoantigens derived from missense mutations of MSI-H patients were calculated and compared to frameshift-derived epitope load. Despite the fact that total frameshift and missense mutation loads are similar, the amount of MHC-I epitopes per mutation were different: 4 epitopes per frameshift and 2—per one missense mutation on average. While most of missense-derived epitopes were matched well with derived normal protein sequences, the majority of frameshift-derived epitopes were unique, “non-human” peptide sequences. This implies that frameshift-derived epitopes have never been seen by the immune system, and targeted T-cells may represent none or minimal reactivity to normal epitopes. These two epitope datasets were also compared with virus-derived antigens. At different search parameters, the overall amount of missense epitopes, matched with viral ones, was higher than frameshift epitopes. This indicates overall viral adaptation to the human proteome, thus helping virus to hijack particular host functionalities and avoid immune system recognition at the same time.

### Identification of Frameshift (Fs) Peptides by Tandem Mass Spectrometry (MS/MS)

Analysis of available MS/MS datasets derived from MSI-H cancer cell lines and patient tumor samples was performed to validate expression of predicted fs peptides at protein level. An available MS/MS search engine, PepQuery (Wen et al. (2019) Genome Res. 29:485-493), allows performance of peptide spectrum match (PSM) for a peptide of interest in the experimental MS/MS spectra dataset. Briefly, using a target peptide sequence, PepQuery retrieves candidate MS/MS spectra which then undergo multiple steps of filtering and statistical evaluations in order to find the best matching MS/MS spectra to the queried peptide. The resulting output is the statistically (Hyperscore, p-value) best-matching MS/MS spectrum which can be visualized using a proteomics data viewer, like PDV (Li et al. (2019) Bioinformatics 35:1249-1251).

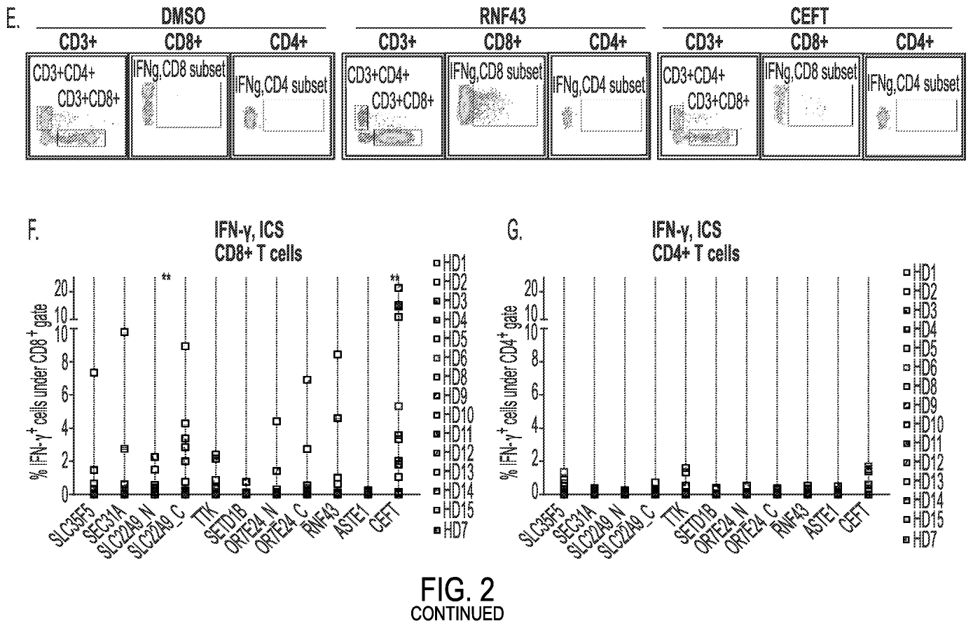

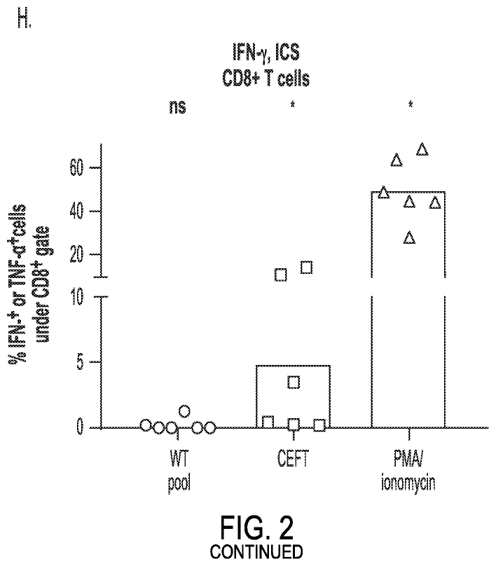

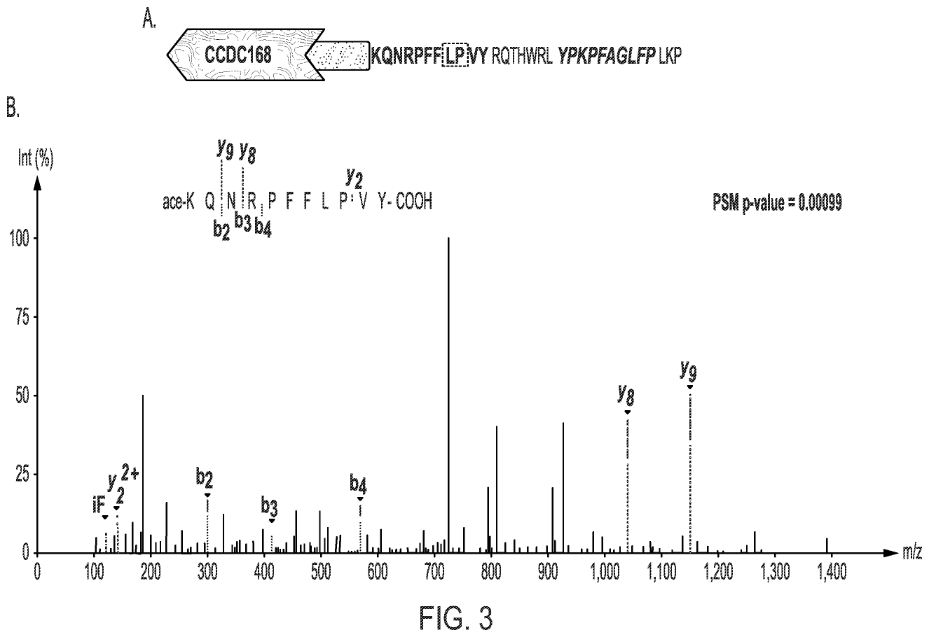

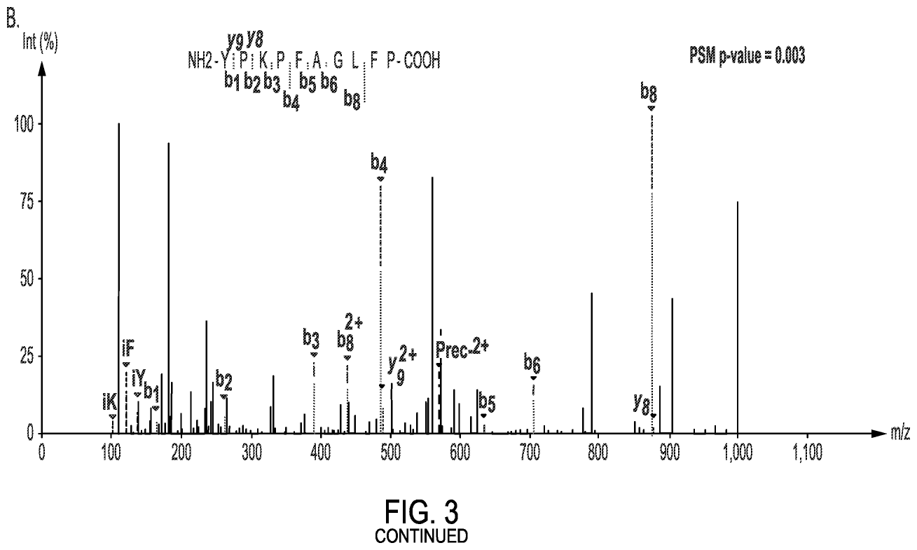

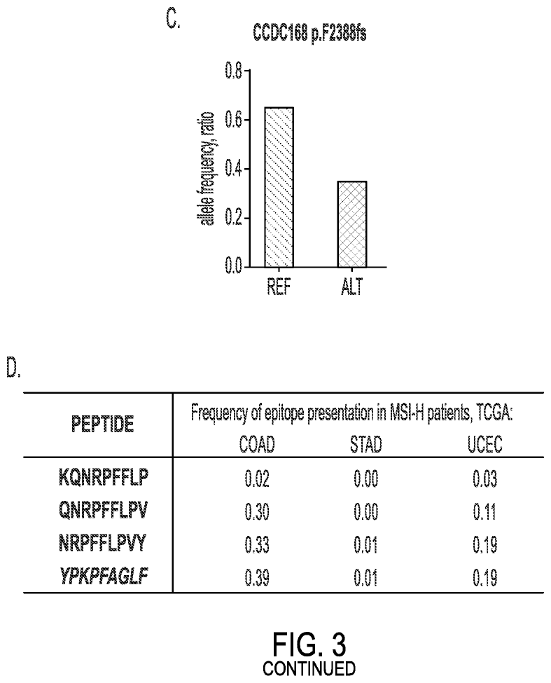

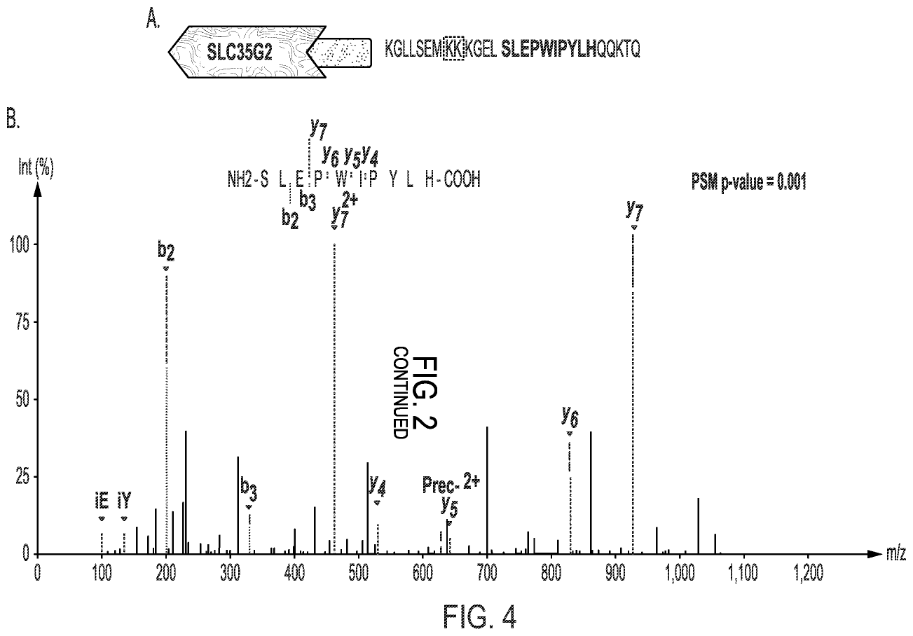

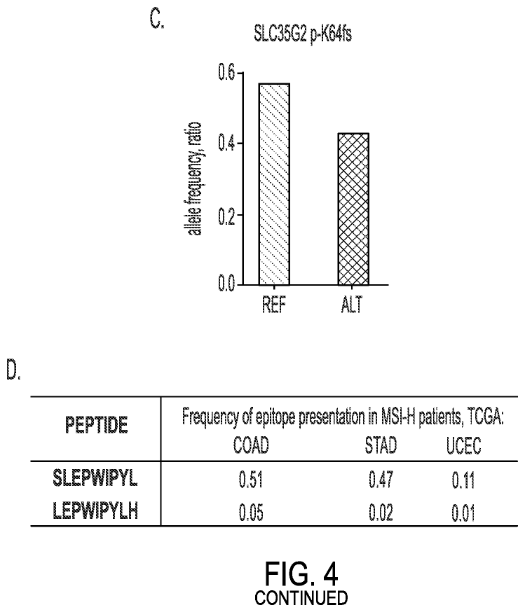

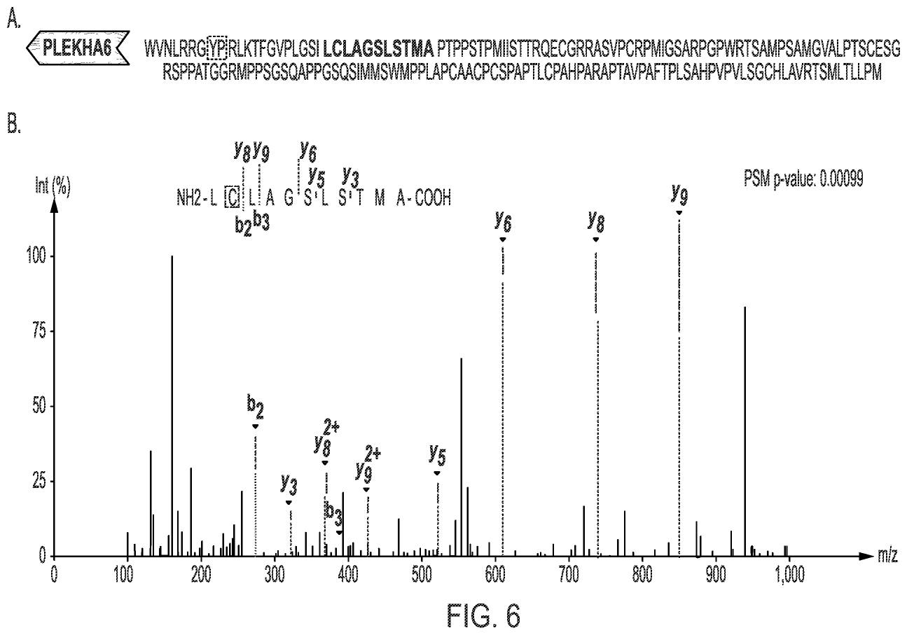

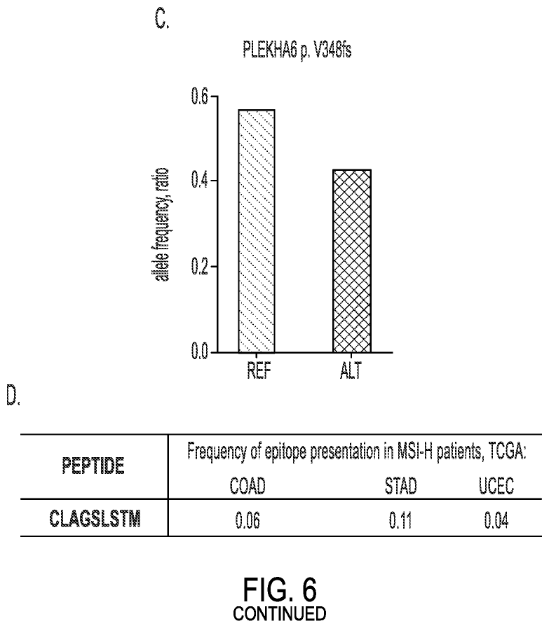

First, the presence of shared fs epitopes in a MS/MS dataset of peptides eluted from MHC-I complexes of HCT116 cancer cell line, derived from a patient with MSI-H colon tumor was analyzed. A few epitopes derived from a predicted fs peptide were detected in MHC-I eluates (FIGS. 3-5). As an example, the predicted fs peptide derived from indel mutation in CCDC168 gene (FIG. 3A) has two MHC-I epitopes detected by MS/MS with p-values 0.00099 and 0.003 respectively: KQNRPFFLPVY (SEQ ID NO: 151) and YPKPFAGLFP (SEQ ID NO:152) (FIG. 3B. The presence of the predicted frameshift is also supported on genomic level by the indel frequency estimated from a whole exome sequencing (WES) experiment retrieved from the CCLE database. As shown on in FIG. 3C, the tumor-specific indel frequency which generates the predicted frameshift (p.F2388fs) is ˜0.35. Finally, the frequency of 9-mer epitopes, overlapping with MS/MS supported fs epitopes to be presented by MHC-I of MSI-H patients, was analyzed. As shown in FIG. 3D, MS/MS-supported epitopes are restricted by frequent HLA-alleles in the MSI-H patient population of The Cancer Genome Atlas (TCG dataset. Similar conclusions can be drawn from SLC35G2 and PLEKHA6 fs peptide analyses (FIGS. 4 and 6).

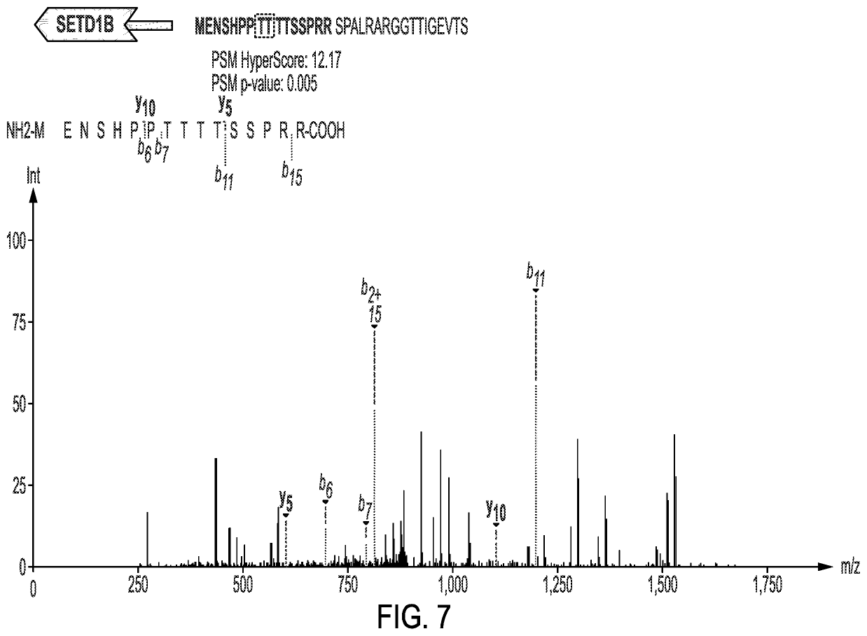

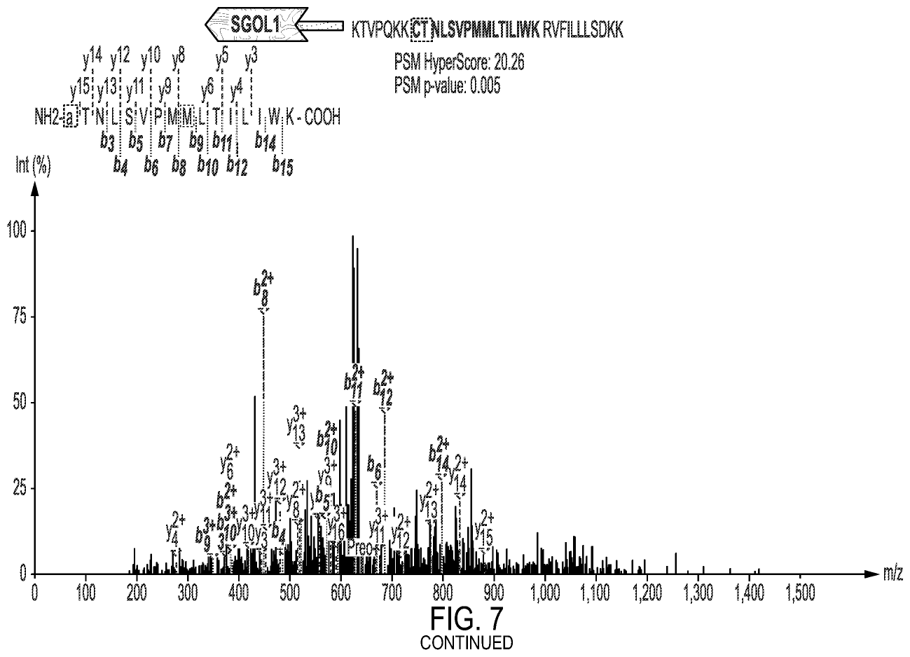

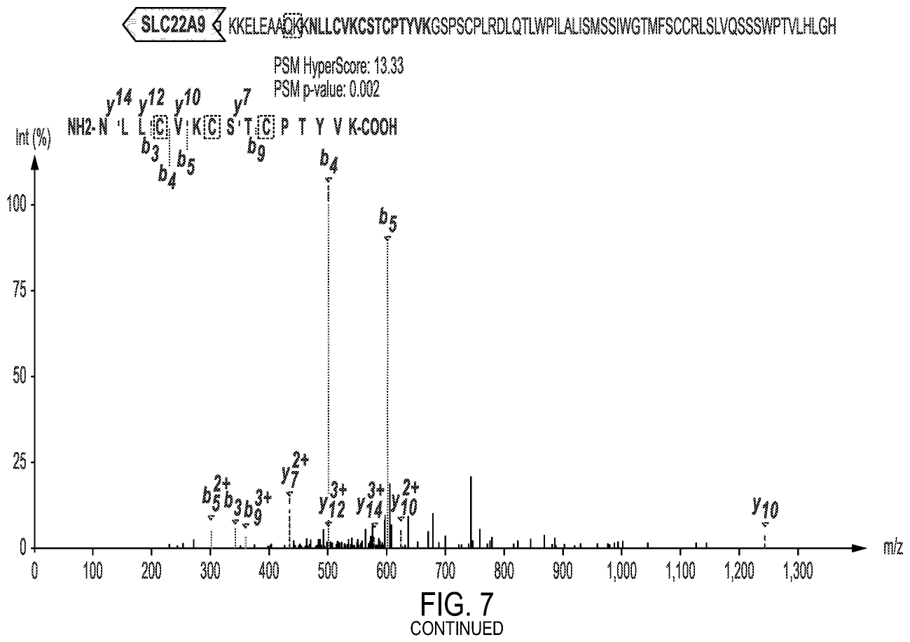

Next, it was determined whether fs peptides can detected in patients' MSI-H tumors. The PepQuery pipeline was applied to whole cell MS/MS datasets retrospectively collected from TCGA colorectal tumors (Zhang et al. (2014) Nature 513:382-387). Experimentally, whole cell protein extracts were processed with trypsin, and the pipeline was adjusted to detect tryptic peptides derived from predicted fs peptides. As an example, several tryptic peptides derived from three fs peptides were identified by MS/MS in a TCGA-AA-AOOR COAD MSI-H tumor sample (FIG. 7).

FIG. 3 shows data from MHC-I epitopes predicted from frameshift peptides that are presented by HCT116 cell line and derived from colon cancer with MSI-H genotype. FIG. 3A shows the schema of the predicted frameshift peptide of SEQ ID NO:23. Epitopes eluted from MHC-I and identified by MS/MS are KQNRPFFLPVY and YPKPFAGLFP. The position of the frameshift mutation within the peptide sequence is amino acids 8 and 9 (LP). FIG. 3B shows MS/MS spectra of MHC-I epitopes by PepQuery. Statistical significance of the peptide-spectrum match (PSM) is defined by p-value. FIG. 3C shows reference and alternative allele frequencies in HCT116 cell line as estimated by WES and/or RNAseq experiments derived from Cancer Cell Line Encyclopedia (CCLE). FIG. 3D shows frequency of 9-mer epitope presentation in MSI-H patient cohorts, Tumor Cancer Genome Atlas (TCGA) dataset. 9-mer epitopes are derived from antigens identified in MS/MS spectra.

FIG. 4 shows data from MHC-I epitopes predicted from frameshift peptides that are presented by HCT116 cell line and derived from colon cancer with MSI-H genotype. FIG. 4A shows the schema of the predicted frameshift peptide of SEQ ID NO:21. An epitope eluted from MHC-I and identified by MS/MS is SLEPWIPYLH. The position of the frameshift mutation within the peptide sequence is amino acids 8 and 9 (KK). FIG. 4B shows MS/MS spectra of MHC-I epitopes by PepQuery. Statistical significance of the peptide-spectrum match (PSM) is defined by p-value. FIG. 4C shows reference and alternative allele frequencies in HCT116 cell line as estimated by WES and/or RNAseq experiments derived from Cancer Cell Line Encyclopedia (CCLE). FIG. 4D shows frequency of 9-mer epitope presentation in MSI-H patient cohorts, Tumor Cancer Genome Atlas (TCGA) dataset. 9-mer epitopes are derived from antigens identified in MS/MS spectra.

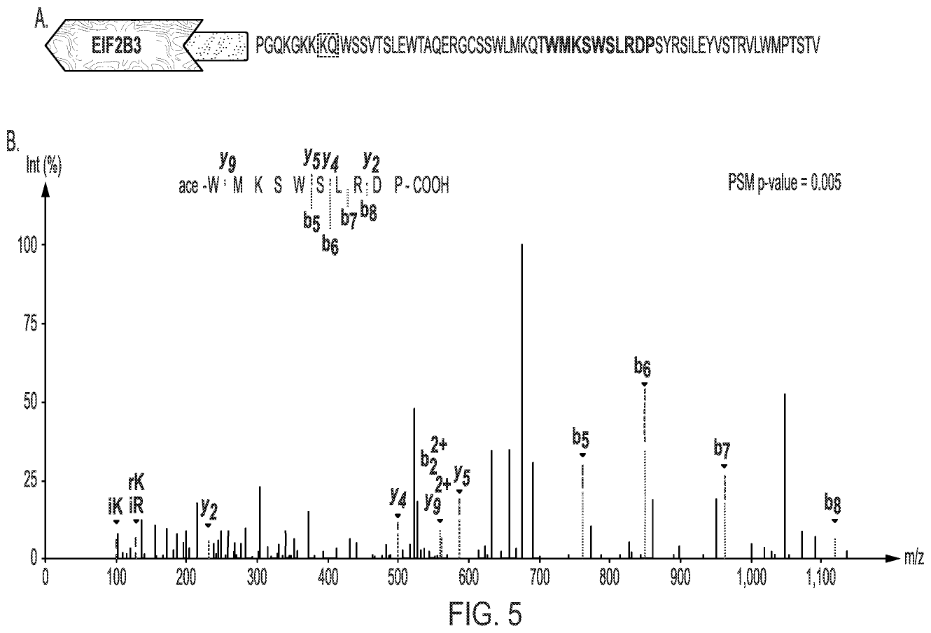

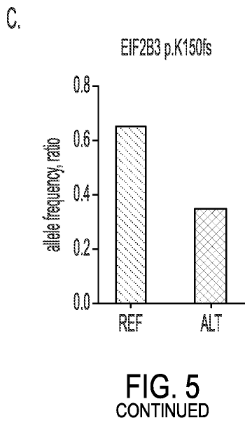

FIG. 5 shows data from MHC-I epitopes predicted from frameshift peptides that are presented by HCT116 cell line and derived from colon cancer with MSI-H genotype. FIG. 5A shows the schema of the predicted frameshift peptide of SEQ ID NO:32. An epitope eluted from MHC-I and identified by MS/MS is WMKSWSLRDP. The position of the frameshift mutation within the peptide sequence is amino acids 8 and 9 (KQ). FIG. 5B shows MS/MS spectra of WIC-I epitopes by PepQuery. Statistical significance of the peptide-spectrum match (PSM) is defined by p-value. FIG. 5C shows reference and alternative allele frequencies in HCT116 cell line as estimated by WES and/or RNAseq experiments derived from Cancer Cell Line Encyclopedia (CCLE).

FIG. 6 shows data from MHC-I epitopes predicted from frameshift peptides that are presented by HCT116 cell line and derived from colon cancer with MSI-H genotype. FIG. 6A shows the schema of the predicted frameshift peptide of SEQ ID NO:45. An epitope eluted from MHC-I and identified by MS/MS is LCLAGSLSTMA. The position of the frameshift mutation within peptide sequence is amino acids 8 and 9 (YP). FIG. 4B shows MS/MS spectra of MHC-I epitopes by PepQuery. Statistical significance of the peptide-spectrum match (PSM) is defined by p-value. FIG. 4C shows reference and alternative allele frequencies in HCT116 cell line as estimated by WES and/or RNAseq experiments derived from Cancer Cell Line Encyclopedia (CCLE). FIG. 4D shows frequency of 9-mer epitope presentation in MSI-H patient cohorts, Tumor Cancer Genome Atlas (TCGA) dataset. 9-mer epitopes are derived from antigens identified in MS/MS spectra.

FIG. 7 shows MS/MS spectra of predicted frameshift peptides in whole-cell MS/MS experiment from TCGA-AA-AOOR COAD MSI-H tumor sample, Clinical Proteomic Tumor Analysis Consortium dataset (CPTAC). Predicted frameshift peptides for SEQ ID NOs: 27, 25, and 29 are represented in schema. The tryptic peptide for SEQ ID NO:25 is CTNLSVPMMLTILIWK and the frameshift mutation is at amino acid positions 8 and 9. The tryptic peptide for SEQ ID NO:9 is NLLCVKCSTCPTYVK and the frameshift mutation is at amino acid positions 8 and 9. Statistical significance of peptide-spectrum match by PepQuery: p-value and hyperscore, are shown under the peptide schema. PSM MS/MS spectra identified by PepQuery for each represented peptide is shown on the right.

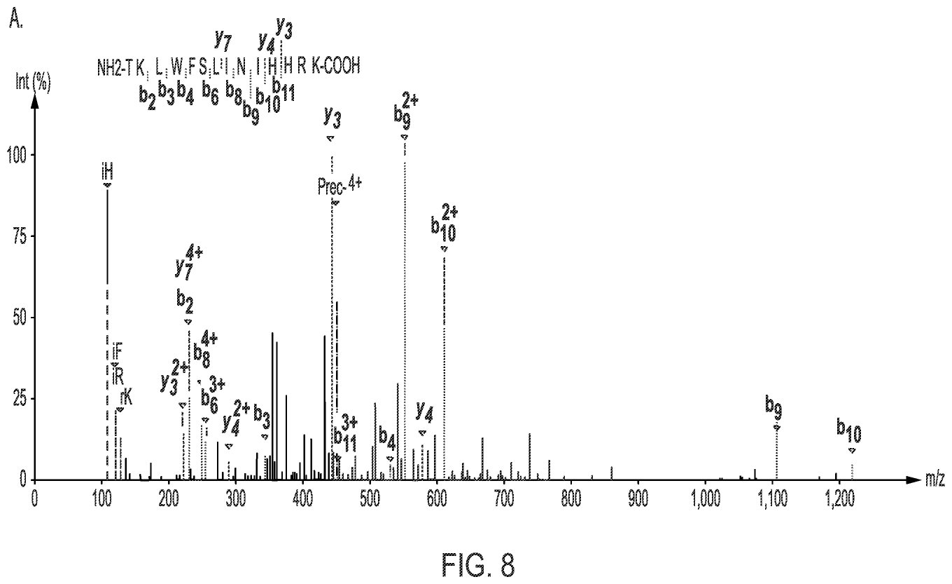

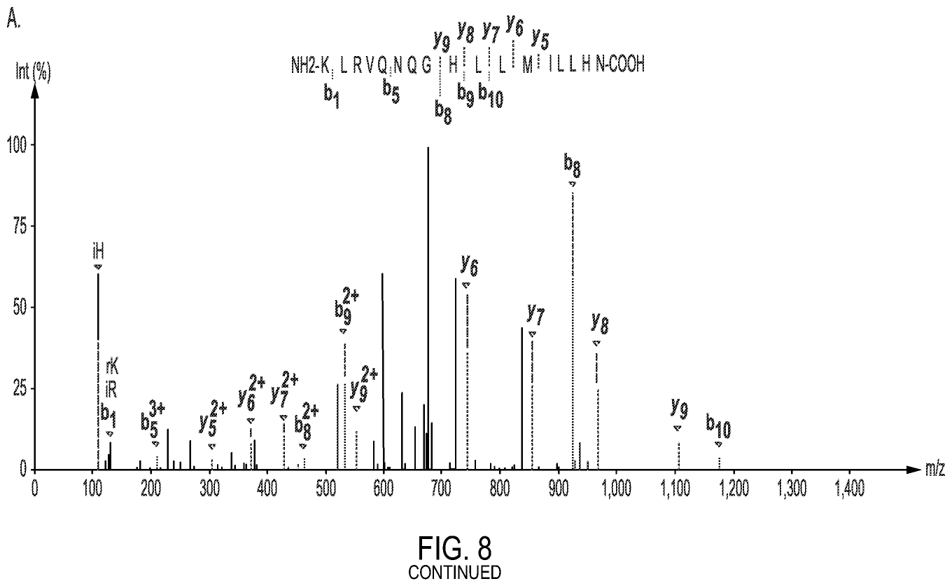

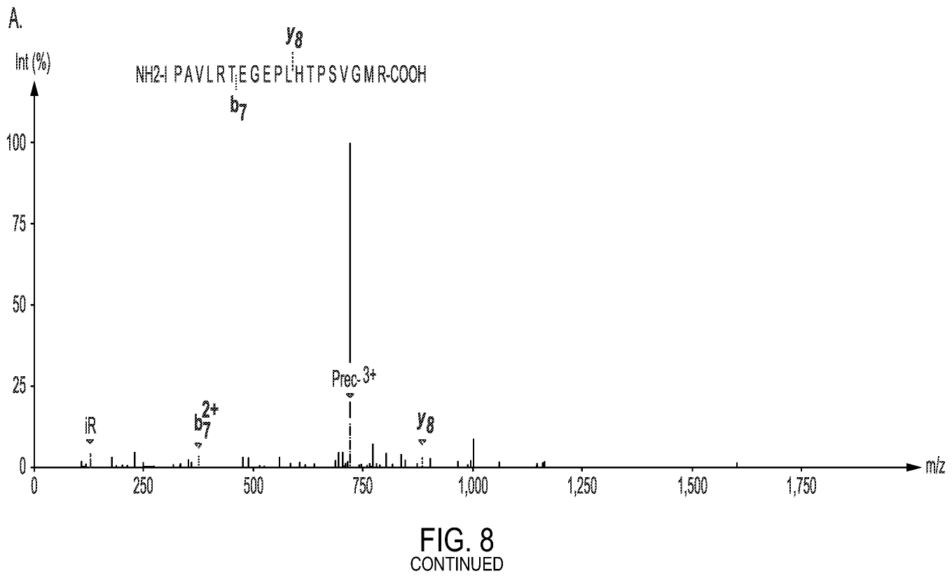

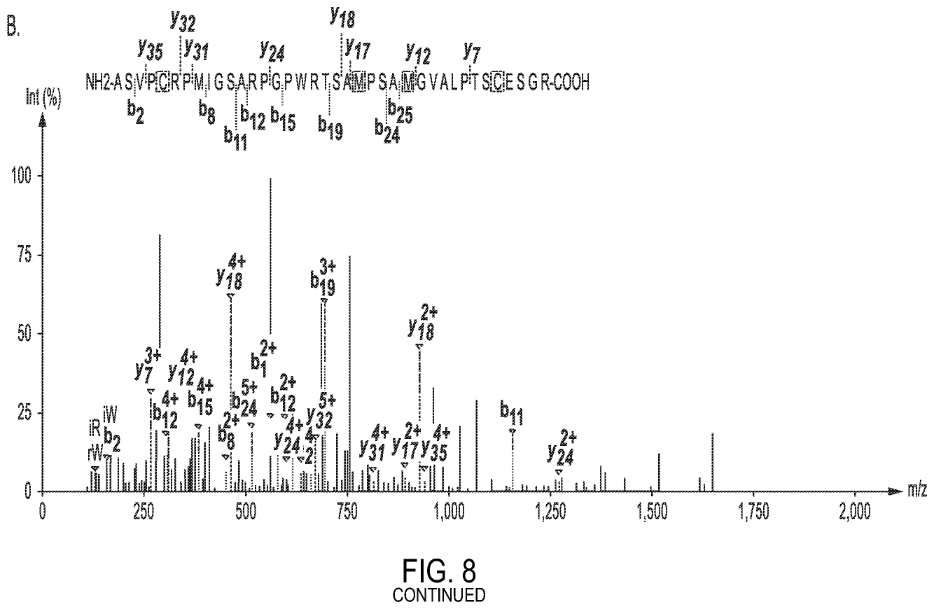

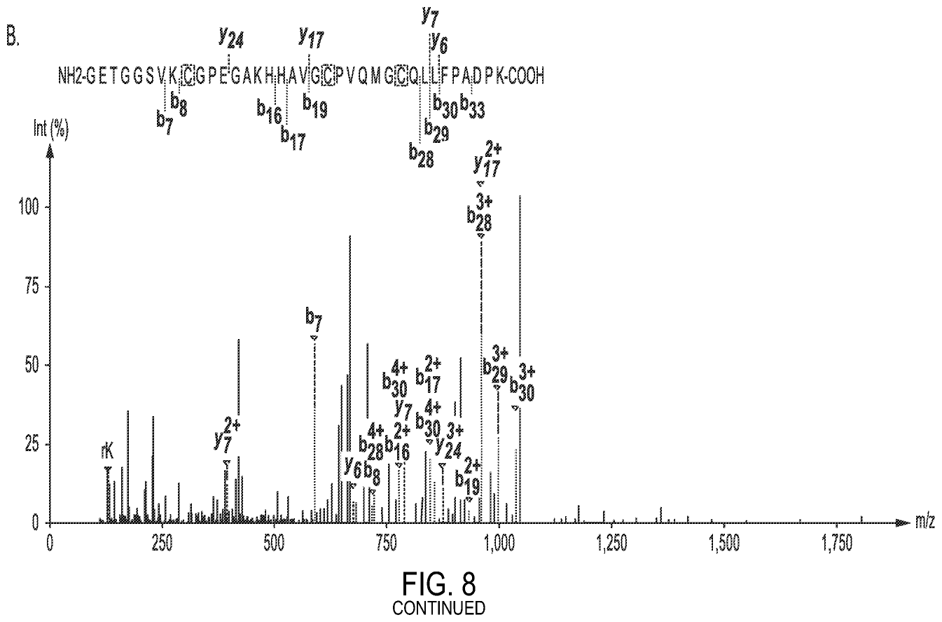

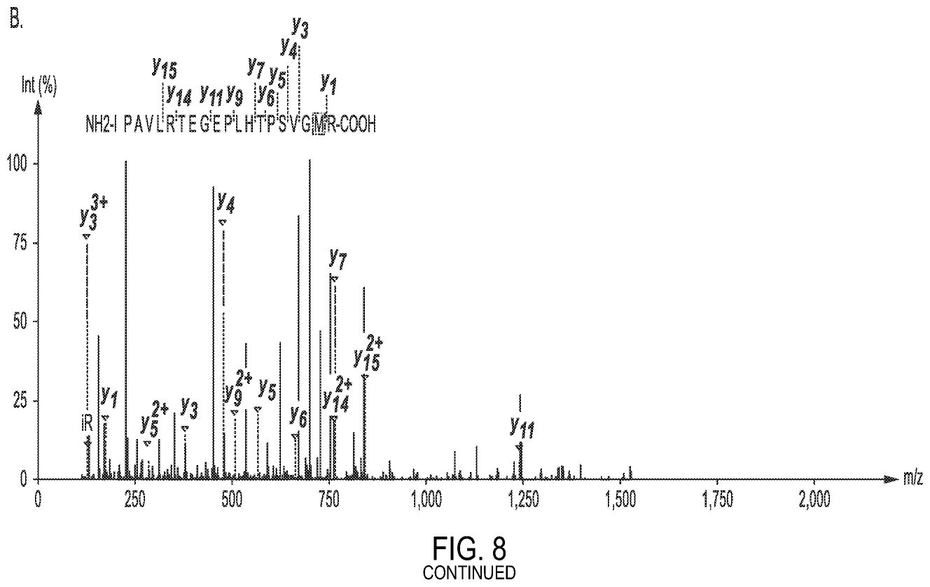

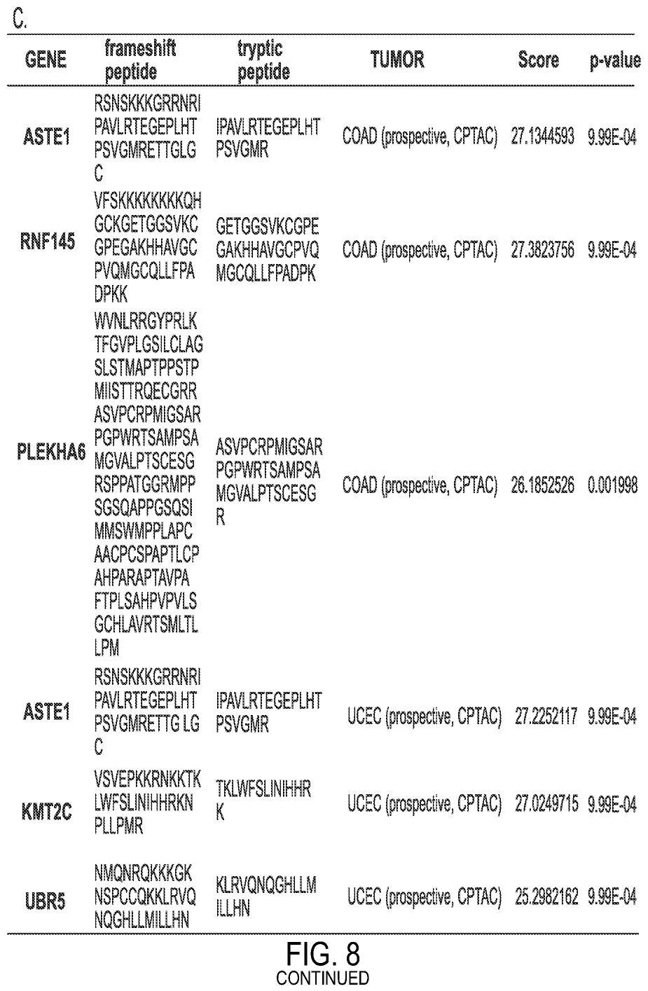

FIG. 8 shows MS/MS spectra of predicted frameshift peptides in whole-cell MS/MS experiment from TCGA-AA-AOOR COAD MSI-H tumor sample, Clinical Proteomic Tumor Analysis Consortium dataset (CPTAC). Predicted frameshift peptides for SEQ ID NOs: 27, 25, and 29 are represented in schema. The tryptic peptide for SEQ ID NO:25 is CTNLSVPMMLTILIWK and the frameshift mutation is at amino acid positions 8 and 9. The tryptic peptide for SEQ ID NO:9 is NLLCVKCSTCPTYVK and the frameshift mutation is at amino acid positions 8 and 9. Statistical significance of peptide-spectrum match by PepQuery: p-value and hyperscore, are shown under the peptide schema. PSM MS/MS spectra identified by PepQuery for each represented peptide is shown on the right.

### Experimental Procedures

### Computational Analysis of TCGA Data

Tumor-associated antigens were predicted using somatic mutation datasets, called by internal mutation pipelines of TCGA. Briefly, annotated somatic missense and frameshift mutations by Mutect2, Somatic Sniper, Varscan and Muse were combined together per each patient. In case of somatic missense mutations, corresponding 17-amino acid residue-length normal peptides, surrounding mutation site, were converted to tumor-specific peptides and used for MHC-I epitope prediction. In case of frameshift mutations, the tumor specific peptide was called as follows: major mRNA isoform was mutated according to the frameshift mutation, translated starting from “−8” amino acid residue position from the mutation site until the stop codon within the new open reading frame, defined by the frameshift. Resulting frameshift peptides were used for MHC-I epitope prediction. NetMHC v4.0 and NetMHCpan v3.08,7 were used to predict missense and frameshift epitopes. HLA allele types for >5000 patients from TCGA were taken from Charoentong, P. et al. Cell Reports. 18:248-262 (2017). Collected epitope data was analyzed using statistical packages, available in Prism and R, using custom written scripts.

### Rapid T-Cell Activation Protocol

Healthy donor PBMCs were cultured in X-VIVO15 media with GM-CSF (1000 IU/mL), IL-4 (500 IU/mL) and Flt3L (50 ng/mL) overnight and then stimulated with peptides (1 μg/mL) in the presence of LPS (100 ng/mL), R848 (10 μM) and IL-1β (5 μg/mL) in X-VIVO15. Long overlapping peptides (15 amino acids) encompassing each mutated protein were pooled together (3-12 peptides/pool). The next day, cells were fed with IL-2 (10 IU/mL) and IL-7 (10 ng/mL) in RPMI media containing 10% human serum. Cells were fed every 2-3 days. IL-2 and IL-7 were not added at the last feeding. After 10 days of culture, cells were harvested and re-stimulated with peptides (1 μg/mL) in the presence of anti-CD28 (0.5 mg/mL) and anti-CD49d (0.5 mg/mL) antibodies. IFN-γ formation was measured by flow cytometry or ELISPOT. For flow cytometry, 1 hour after re-stimulation with peptides, cells were added BD GolgiStop™, containing monensin and BD GolgiPlug™, containing brefeldin A according to the manufacturer's suggestion. IFN-γ production was measured 12-hours after the addition of protein transport inhibitors by intracellular staining using BD Cytofix/Cytoperm™ reagents according to manufacturer's protocol. For ELISPOT analysis, cells were re-stimulated in plates with mixed cellular ester membrane that were coated with anti-IFN-γ antibody (4 μg/mL). Plates were processed for IFN-γ detection after 48-hours of culture.

### MS/MS Analysis

MS/MS datasets were downloaded from PRIDE (https://www.ebi.ac.uk/pride/archive/) or CPTAC (https://proteomics.cancer.gov/data-portal) repositories. Retrieved data was analyzed using PepQuery (http://www.pepquery.org). Briefly, raw MS/MS spectra was converted to MGF format using msconvert (http://proteowizard.sourceforge.net/tools.shtml), which was then supplied to command-line installed PepQuery. In case of the analysis of the HCT116 MHC-I MS/MS dataset, predicted fs peptides were computationally sliced on overlapping 8-, 9-, 10- and 11-mer epitopes. The produced list of epitopes was submitted to PepQuery analysis.

### Example 2—Selected MSI-H Endometrial Tumor-Specific Frameshift Peptides are Highly Immunogenic

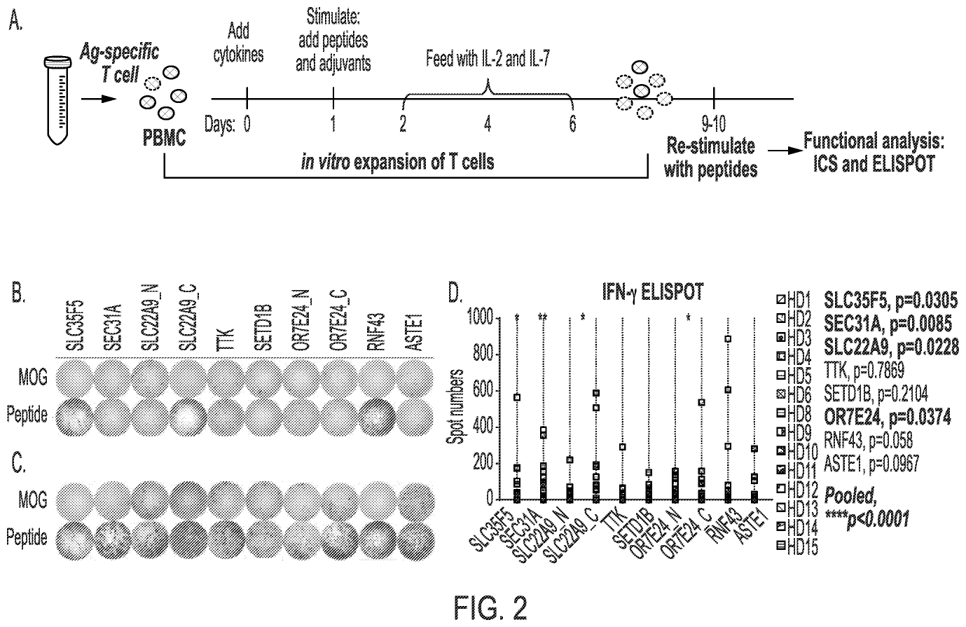

To assess the immunogenicity of the nine predicted fs-peptides from MSI-H UCEC patient cohort, the T cell responses against each neopeptide were evaluated using a T cell immunogenicity assay that is designed to rapidly prime naïve T cells. In brief, long overlapping peptide (OLP) libraries spanning each fs-peptide were designed. Using these OLP pools, T cells from 15 randomly picked healthy donors (HD) were primed and expanded. After expansion, the cells were stimulated with the OLP pools and fs-peptide-specific T cell responses were evaluated by measuring IFN-γ production using ELISPOT (FIG. 2A). Results showed that each fs-peptide could elicit T cells responses in a subset of subjects tested. Furthermore, some subjects had reactive T cells against multiple fs-peptides (FIGS. 2B-D). When combined, the fs-peptide-specific T cells were significantly enriched in the subject cohort. The fs-peptide-specific T cell responses in the same HD cohort were also characterized by intracellular staining (ICS). Results from both assays showed similar stimulation profiles. Moreover, responses to fs-peptides were observed primarily in CD8+ T cells, reaching up to 10% of T cells indicating strong priming to these neoantigens. (FIGS. 2E-G). In total, a majority of HD (13 out of 15) responded to at least one fs-peptide. The reactive T cells produced TNF-α, in addition to IFN-γ, indicating that fs-peptide-specific T cells are polyfunctional. Additionally, control peptides (15-aa) were synthesized for each fs-peptide using their wild type sequence surrounding the fs-mutation site. Responses by HD T cells to stimulation with WT OLP pool were not higher than the background (FIG. 2H), indicating that the observed T cell responses were specific to fs-peptides. Next, it was investigated whether the fs-peptide-specific T cells responses that were observed in the HD cohort correlated with the predicted high affinity epitope load. To determine the predicted epitope load, the HLA-I alleles of each subject were identified by sequence-based HLA-I genotyping and the predicted binding affinity of epitopes from fs-peptides to each subject's unique HLA was investigated. No significant correlation between the total epitope load per patient and experimentally observed response rate was found. Altogether, the foregoing data show that MSI high patients have an increased frequency of high-quality T cell epitopes derived from shared fs-peptides, binding to a broad spectrum of HLA alleles, capable of inducing immunogenicity for CD8+ T cell in particular.

### Other Embodiments

Any improvement may be made in part or all of the peptides, vaccines, compositions, kits, and method steps. All references, including publications, patent applications, and patents, cited herein are hereby incorporated by reference in their entireties. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A tumor vaccine comprising at least one neoantigenic peptide having the amino acid sequence of one of SEQ ID NOs:1-69 or an immunogenic fragment thereof, and an adjuvant.

2. The tumor vaccine of claim 1 wherein the at least one neoantigenic peptide has the amino acid sequence of one of SEQ ID NOs:2, 3, 6, 8, and 47 or an immunogenic fragment thereof.

3. The tumor vaccine of claim 2 comprising neoantigenic peptides having the sequences of SEQ ID NOs:2, 3, 6, 8, and 47 or immunogenic fragments thereof.

4. The tumor vaccine of claim 1, wherein the tumor is a (microsatellite instability hypermutated) MSI-H endometrial tumor, and the at least one neoantigenic peptide has the amino acid sequence of one of SEQ ID NOs:1-9 or an immunogenic fragment thereof.

5. The tumor vaccine of claim 1, wherein the tumor is a MSI-H colorectal tumor, and the at least one neoantigenic peptide has the amino acid sequence of one of SEQ ID NOs:10-46 or an immunogenic fragment thereof.

6. The tumor vaccine of claim 1, wherein the tumor is a MSI-H stomach tumor, and the at least one neoantigenic peptide has the amino acid sequence of one of SEQ ID NOs:47-69 or an immunogenic fragment thereof.

7. The tumor vaccine of claim 1, wherein the at least one neoantigenic peptide is a plurality of neoantigenic peptides.

8. A method of inducing or enhancing an immune response in a subject having a MSI-H tumor or at risk of having an MSI-H tumor, comprising administering to the subject a therapeutically effective amount of the tumor vaccine of claim 1.

9. The method of claim 8, wherein the subject has a MSI-H endometrial tumor or is at risk of having a MSI-H tumor, and the tumor vaccine comprises at least one neoantigenic peptide having the amino acid sequence of one of SEQ ID NOs:1-9 or an immunogenic fragment thereof.

10. The method of claim 8, wherein the subject has a MSI-H colorectal tumor or is at risk of having a MSI-H colorectal tumor, and the tumor vaccine comprises at least one neoantigenic peptide having the amino acid sequence of one of SEQ ID NOs:10-46 or an immunogenic fragment thereof.

11. The method of claim 8, wherein the subject has a MSI-H stomach tumor or is at risk of having a MSI-H stomach tumor, and tumor vaccine comprises at least one neoantigenic peptide having the amino acid sequence of one of SEQ ID NOs:47-69 or an immunogenic fragment thereof.

12. The method of claim 8 wherein the vaccine comprises a plurality of neoantigenic peptides or immunogenic fragments thereof.

13. The method of claim 8 wherein the subject is a human subject.

14. An isolated peptide having an amino acid sequence selected from the sequences of SEQ ID Nos:1-69

15. Use of a tumor vaccine comprising at least one neoantigenic peptide having the amino acid sequence of one of SEQ ID NOs:1-69 or an immunogenic fragment thereof, for treatment of a MSI-H tumor.

16. A pharmaceutical composition for use in adoptive cell therapy to treat a tumor comprising a population of T-cells expressing one or more chimeric antigen receptors (CARs) or one or more T-cell receptors (TCRs) that are reactive to at least one neoantigenic peptide having the amino acid sequence of one of SEQ ID NOs:1-69 or a fragment thereof.

17. The pharmaceutical composition of claim 16, wherein the at least one neoantigenic peptide has the amino acid sequence of one of SEQ ID NOs:2, 3, 6, 8, and 47 or a fragment thereof.

18. The pharmaceutical composition of claim 16, wherein the tumor is a MSI-H endometrial tumor, and the at least one neoantigenic peptide has the amino acid sequence of one of SEQ ID NOs:1-9 or a fragment thereof.

19. The pharmaceutical composition of claim 16, wherein the tumor is a MSI-H colorectal tumor, and the at least one neoantigenic peptide has the amino acid sequence of one of SEQ ID NOs:10-46 or a fragment thereof.

20. The pharmaceutical composition of claim 16, wherein the tumor is a MSI-H stomach tumor, and the at least one neoantigenic peptide has the amino acid sequence of one of SEQ ID NOs:47-69 or a fragment thereof.

21. A method of inducing or enhancing an immune response in a subject having a MSI-H tumor or at risk of having an MSI-H tumor, comprising administering to the subject a therapeutically effective amount of the pharmaceutical composition of claim 16.
